(19) United States
(12) Patent Application Publication
(10) Pub. No.: US 2013/0095512 A1
(43) Pub. Date: Apr. 18, 2013

(54) DEVICE FOR SORTING, CLASSIFYING, AND ASSAYING PARTITION BEHAVIOR OF CELL MEMBRANE BIOMOLECULES AND METHODS BASED THEREON

(71) Applicants: Susan Daniel, Ithaca, NY (US); Ling Chao, New Taipei City (TW)

(72) Inventors: Susan Daniel, Ithaca, NY (US); Ling Chao, New Taipei City (TW)

(73) Assignee: Cornell University, Ithaca, NY (US)

(21) Appl. No.: 13/805,179

(22) PCT Filed: Jun. 17, 2011
(86) PCT No.: PCT/US11/40962

§ 371 Date: Dec. 18, 2012

Related U.S. Application Data
(60) Provisional application No. 61/356,393, filed on Jun. 18, 2010.

(51) Int. Cl. G01N 33/68; G01N 27/447

(52) U.S. Cl. CPC G01N 33/6842; G01N 27/44791; G01N 27/44782
USPC 435/21; 422/527; 436/34; 436/172; 204/601; 204/451

(57) ABSTRACT

A biomolecule partitioning device (BPD) is provided that can be used to separate and sort membrane species into raft-like membrane regions without using detergent or crosslinkers. The BPD can comprise one or more microfluidic channels coated with coexistent lipid phases (raft-like and fluid-like lipid compositions) as a contiguous supported lipid bilayer (SLB). The geometry of the phases can be patterned with spatial and temporal control within each channel. Methods for the separation and sorting are also provided. The method can comprise the steps of introducing cell membrane species into an SLB; patterning coexistent phases; applying an electric field or hydrodynamic flow to move the species; sorting migrating species into regions based on their partitioning preference; and collecting sorted species in a quantification area. The BPD can also be used to measure partitioning kinetics or to assay for activity changes of biomolecules as a function of local lipid environment.

### CROSS-REFERENCE TO RELATED APPLICATIONS

This application claims priority to and the benefit of co-pending U.S. provisional patent application Ser. No. 61/356,393, entitled “Device For Sorting, Classifying, and Assaying Partition Behavior of Cell Membrane Biomolecules and Methods Based Thereon,” filed Jun. 18, 2010, which is incorporated herein by reference in its entirety.

### STATEMENT REGARDING FEDERALLY SPONSORED RESEARCH OR DEVELOPMENT

The disclosed invention was made with government support under contract no. EEC-0824381 from the National Science Foundation. The government has rights in this invention.

### 1. TECHNICAL FIELD

The present invention relates to devices for separating, sorting, classifying, and/or assaying membrane biomolecules such as cell membrane biomolecules. The invention further relates to methods for separating, sorting, classifying, and assaying membrane biomolecules. The invention also relates to devices and methods for quantifying the interactions of membrane biomolecules with other membrane species. The invention also relates to devices and methods for measuring the rates at which membrane biomolecules associate/dissociate with membrane compositions.

### 2. BACKGROUND OF THE INVENTION

Membrane proteins constitute a significant portion of the human genome (˜35%) and are important targets for disease mitigation. They are the targets of nearly 60-70% of all drug candidates currently being developed. Thus they are of vital interest for a number of industries from pharmaceutical to biotechnology. However, getting information about the structure and function of membrane proteins is extremely difficult and thus severely impacts our basic understanding of these species. This information is needed for a variety of applications from drug discovery to development of protein biosensors for homeland security. The difficulty in studying membrane proteins stems from the inability to preserve their structures outside their native hydrophobic environment. A staggering statistic that captures the paucity of data available on these species is that of the ˜1000 G-protein-linked receptors that have been identified, less than four unique structures have been solved to date. Moreover, structural information is not enough to understand or even predict a protein's function or behavior in an intact cell; mounting data ties protein function to interaction with the membrane itself, including lipids such as phospholipids, cholesterol, and other species. Thus there is a need in the art for in vitro platforms that can aid in understanding membrane species in their own environment which also protects the proteins' hydrophobic cores, ensures their proper orientation for interaction, and safeguards their overall structure, while also being able to assay protein-lipid interactions.

Lipid Rafts

Many proteins are thought to associate with distinct regions in the membrane having different lipid compositions. These associations can help resident proteins carry out their functions and movement between membrane domains can also serve as a means of regulating protein function. This idea is the basis of the “raft hypothesis,” which has postulated that heterogeneous regions in the cell membrane, called rafts, are enriched in sterols and sphingolipids relative to surrounding phospholipids (FIG. 1). These nanometer-scale domains exist to either locally sequester proteins to increase their probability of carrying out some function, such as signaling, or to isolate certain species inside the raft from species outside. Rafts were originally defined based on their solubility with detergent. The proteins associated with these “detergent resistant membrane” (DRM) fractions are now best thought of as “candidates” to be associated with rafts, because there is significant evidence that many proteins found in DRMs are contaminants and do not associate with rafts in intact cells. Detergents can artifactually cause disparate membrane components to coalesce. Furthermore, visualization of rafts has typically been performed on fixed cells, leading to possible cross-linking artifacts. Most efforts to observe rafts in live cells have used proteins that can also cross-link (such as cholera toxin's B subunit). Other attempts have utilized fluorescently-labeled lipid analogs, but these have been shown to have different behavior than endogenous lipids. Thus, it is technically difficult to observe and understand raft-related events.

Lipid rafts in the cell membrane are thought to play a crucial role in signal transduction, its regulation, and the manifestation of certain diseases. Raft proteomics and lipidomics focuses on discovering and classifying the types of proteins and biomolecules constituting rafts and their partition response to a biological or chemical stimulus. However, current approaches to define the raft proteome are based on the use of detergent or crosslinking agents, which can artifactually cause disparate membrane components to coalesce and lead to erroneous classifications. There is therefore a need in the art for devices to sort membrane species into raft-like regions without using detergent or crosslinkers. There is also a need in the art for assays that can facilitate the proteomic identification of raft residents for many species to aid in understanding regulation of cell biology of the membrane and its regulation. This type of assay should be of broad interest to many industries that focus on membrane proteomics.

Current understanding of the cell membrane suggests that it is a patchwork structure composed of many proteins and lipids that are not all freely diffusing, but rather can take part in dynamic microdomains within the plane of the membrane. These domains can form or be maintained in several ways, such as “lipid shells” around proteins and/or cytoskeletal compartmentalization (Kusumi A, Koyama-Honda I, Suzuki K. Molecular dynamics and interactions for creation of stimulation-induced stabilized rafts from small unstable steady-state rafts. Traffic. 2004; 5:213-30; Edidin M. Lipids on the Frontier: A Century of Cell-Membrane Bilayers. Nature. 2005; 4:414-8; Simons K, Ikonen E. Functional Rafts in Cell Membranes. Nature. 1997; 387:569-72; Sprenger R R, Horrevoets J G. The Ins and Outs of Lipid Domain Proteomics. Proteomics. 2007; 7:2895-903; Shaw A S. Lipid Rafts: Now You See Them, Now You Don't. Nature Immunology. 2006; 7:1139-42; Zheng Y Z, Foster L J. Biochemical and Proteomic Approaches for the Study of Membrane Microdomains. Proteomics. 2009; 72:12-22; Simons K, Vaz W L C. Model Systems, Lipid Rafts, and Cell Membranes. Annu Rev Biophys Biomol Struct. 2004; 33:269-95). In either case, it is hypothesized that interactions within a micro-environment can not only co-localize multiple components of some functional unit, but also be involved in the regulation of that unit's activity. Regulation can be accomplished by the action of a stimulus that changes the partitioning behavior of a particular species in a microdomain, thereby altering a protein-lipid association vital for activity. However, studies of protein-lipid associations and their impacts on protein activity are currently limited.

A direct strategy for studying impact of lipid-protein interactions in cellular processes would be to knock-out the expression of a specific lipid in the cell membrane. However, it is currently impossible to do this because lipids are typically synthesized by the sequential activities of multiple enzymes in complex synthetic pathways. Although individual enzymes in these pathways can be genetically deleted, there will be changes in all lipids “downstream” of that enzyme in the pathway. Alternatively, one can modulate the amounts of certain lipids in membranes through exogenous addition, or by extraction, or by using enzyme inhibitors to shift equilibria between lipid species, but none of these strategies are ideal for investigating specific interactions.

Researchers have tried to correlate regions in the cell membrane rich in certain lipids with the function of proteins resident in these regions. This strategy has been used most commonly for investigations of “membrane rafts,” which are highly dynamic regions of the membrane that are enriched in sterols and sphingolipids as opposed to phospholipids (Simons K, Ikonen E. Functional Rafts in Cell Membranes. Nature. 1997; 387:569-72; Sprenger R R, Horrevoets J G. The Ins and Outs of Lipid Domain Proteomics. Proteomics. 2007; 7:2895-903; Shaw A S. Lipid Rafts: Now You See Them, Now You Don't. Nature Immunology. 2006; 7:1139-42; Zheng Y Z, Foster L J. Biochemical and Proteomic Approaches for the Study of Membrane Microdomains. Proteomics. 2009; 72:12-22; Pike L J. Rafts defined. J Lipid Research. 2006; 47:1597-8; Simons K, Vaz W L C. Model Systems, Lipid Rafts, and Cell Membranes. Annu Rev Biophys Biomol Struct. 2004; 33:269-95). Approaches to identify and correlate functional dependence of residents of these rafts regions have led to controversial results. Current methods use detergent or high salt/alkaline pH to isolate insoluble membrane fractions (Sprenger R R, Horrevoets J G. The Ins and Outs of Lipid Domain Proteomics. Proteomics. 2007; 7:2895-903; Zheng Y Z, Foster L J. Biochemical and Proteomic Approaches for the Study of Membrane Microdomains. Proteomics. 2009; 72:12-22). Raft residents are then identified by mass spectrometry (MS) or gel electrophoresis and immunoblotting (Foster L J, Chan Q W T. Lipid raft proteomics: more than just detergent-resistance membranes. Bertrand E, Faupel M, editors: Springer; 2007). However, the processing conditions can lead to artifacts, such as variations in compositions depending on conditions or contamination with species from other cellular compartments, which raise the possibility that these fractions coalesced during processing (Sprenger R R, Horrevoets J G. The Ins and Outs of Lipid Domain Proteomics. Proteomics. 2007; 7:2895-903; Zheng Y Z, Foster L J. Biochemical and Proteomic Approaches for the Study of Membrane Microdomains. Proteomics. 2009; 72:12-22). Beyond this, the division of membranes into “raft vs. non-raft” encourages an over-simplified view of biological membranes that overlooks the potential existence of multiple raft sub-types in a single cell (Asano A, Selvaraj V, Bunke D E, Nelson J L, Green K M, Evans J E, et al. Biochemical characterization of membrane fractions in murine sperm: Identification of three distinct sub-types of membrane rafts. J Cell Physiol. 2009; 218:537-48). Thus, there is a need for a device where the specific interaction of biomolecules with a variety of membrane compositions, in one instance, raft compositions, can be investigated.

Other methods to identify microdomain residents involve the direct labeling of the intact cell membrane (Zheng Y Z, Foster L J. Biochemical and Proteomic Approaches for the Study of Membrane Microdomains. Proteomics. 2009; 72:12-22; Munro S. Lipid Rafts: Elusive or Illusive? Cell. 2003; 115:377-88). Surface labeling of cells requires antibodies or toxins to bind to specific species, often crosslinking them and causing artifactual enrichment (Dietrich C, Volovyk Z N, Levi M, Thompson N L, Jacobson K. Partitioning of Thy-1, GM1, and Cross-linked Phospholipid Analogs into Lipid Rafts Reconstituted in Supported Model Membrane Monolayers. Proc Natl Acad Sci. 2001; 98:10642-7). An alternative is to label fixed cells, but membrane organization and protein-lipid associations of dead cells are not necessarily indicative of live conditions (Kusumi A, Suzuki K. Toward understanding the dynamics of membrane-raft-based molecular interactions. Biochim Biophys Acta. 2005; 1746:234-51; Selvaraj V, Asano A, Buttke D E, McElwee J L, Nelson J L, Wollf C A, et al. Segregation of Micron-Scale Membrane Sub-Domains in Live Murine Sperm. J Cell Physiol. 2006; 206:636-46). Isotope labeling methods have some utility (Foster L J, Cheng Q W T. Lipid raft proteomics: more than just detergent-resistance membranes: Springer; 2007), but can only be applied to cultured cells, are expensive, and still require isolation methods to identify residents.

Besides the drawbacks for classifying the intrinsic residents of rafts, many methods have limited or no capability to track dynamic shifts in partitioning of species into or out of rafts or to assay protein activity within these different microenvironments. Changes in partitioning behavior might be initiated by a soluble protein binding to the membrane-bound species (Dietrich C, Volovyk Z N, Levi M, Thompson N L, Jacobson K. Partitioning of Thy-1, GM1, and Cross-linked Phospholipid Analogs into Lipid Rafts Reconstituted in Supported Model Membrane Monolayers. Proc Natl Acad Sci. 2001; 98:10642-7; Kawano N, Yoshida K, Iwamoto T, Yoshida M. Ganglioside GM1 mediates decapacitation effects of SVS2 on murine spermatozoa. Biol Reprod. 2008; 76:1153-9; Wong W, Schlichter L C. Differential recruitment of Kv1.4 and Kv4.2 to lipid rafts by PSD-95. J Biol Chem. 2004; 279:444-52), chemical or drug exposure (Dietrich C, Volovyk Z N, Levi M, Thompson N L, Jacobson K. Partitioning of Thy-1, GM1, and Cross-linked Phospholipid Analogs into Lipid Rafts Reconstituted in Supported Model Membrane Monolayers. Proc Natl Acad Sci. 2001; 98:10642-7; Foster L J, Cheng Q W T. Lipid raft proteomics:more than just detergent-resistance membranes: Springer; 2007; Dolganiuc A, Bakis G, Kodys K, Mandrekar P, Szabo G. Acute ethanol treatment modulates toll-like receptor-4 association with lipid rafts. Alcoholism: Clinical and Experimental Research. 2006; 30:76-85), or changes in ionic strength or pH (Ciana A, Balduini C, Minetti G. Detergent-resistant membrane in human erythrocytes and their connection to the membrane-skeleton. J Biosci. 2005; 30:317-28; Hartmann W, Galla H-J, Sackmann E. Direct evidence of charge-induced lipid domain structure in model membranes. FEBS Letters. 1977; 78:169-72). Thus, there is a need in the art for new approaches that are able to assay protein-lipid interactions after being subjected to stimuli.

Citation or identification of any reference in Section 2, or in any other section of this application, shall not be considered an admission that such reference is available as prior art to the present invention.

### 3. SUMMARY OF THE INVENTION

A biomolecule partitioning device (BPD) for partitioning biomolecules is provided. In one embodiment, the BPD comprises:
a substrate;
a microfluidic channel on the substrate;
a plurality of stable coexistent lipid phases, wherein the surface of the microfluidic channel is coated with a plurality of stable, coexistent lipid phases, thereby forming a supported lipid bilayer.

In another embodiment, the biomolecules are cell membrane species. The cell membrane species can be a protein, a glycolipid, a proteolipid, a proteoglycan or any other membrane species known in the art.

In another embodiment, the plurality of stable coexistent lipid phases comprises a raft-like or a fluid-like lipid composition. Once in the membrane, the biomolecules can partition into either phase.

In another embodiment, the phospholipid-containing region is a raft phase or raft-like region, a 1-Palmitoyl-2-oleoyl-sn-glycero-3-phosphocholine (POPC)-rich region, a cholesterol (Chol)-rich region or a N-palm itoyl-D-erythro-sphingosylphosphorylcholine (PSM)-rich region. The phospholipid-containing region can contain any phospholipid or phospholipid derivative known in the art (e.g.,a phosphatidylcholine (or derivative thereof)). In another embodiment, the region can contain any cholesterol derivative known in the art.

In another embodiment, the plurality of stable coexistent lipid phases are patterned within the microfluidic channel.

In another embodiment, the BPD can comprise a cushion for reducing strong protein interaction with the surface of the microfluidic channel.

In another embodiment, the supported lipid bilayer comprises at least two different stable, coexistent lipid phases in controllable or preselected spatial or temporal geometries.

In another embodiment, an electric field or fluidic flow is applied to the biomolecule.

In another embodiment, the BPD comprises one or more collection channels or ports. In another embodiment, the BPD can comprise one or more loading channels or ports.

In another embodiment, the BPD can comprise two contiguous phases (e.g., FIG. 3) or multiple contiguous phases (e.g., FIG. 7). In other embodiments, the number of phases is 2-10, 10-20, 20-30, 30-40, 40-50, 50-60, 60-70, 70-80, 80-90, 90-100 or greater than 100. The multiple phases can all be unique (e.g., a, b, c, d, e . . . ), alternating repeating (e.g., a, b, a, b, . . . ) or other (e.g., a, b, a, c, a, d . . . or a, b, c, a, d, e . . . ). Such permutations and variations will be apparent to the skilled artisan.

A method for separating biomolecules is also provided. In one embodiment, the method comprises the steps of:
providing a microfluidic channel;
providing a supported lipid bilayer comprising a plurality of stable coexistent lipid phases, wherein the supported lipid bilayer is patterned within the microfluidic channel;
introducing the biomolecules into the supported lipid bilayer;
applying an electric field or hydrodynamic flow to move the biomolecules through the supported lipid bilayer;
separating migrating biomolecules based on their preference for heterogeneous regions in the supported lipid bilayer; and
collecting the separated biomolecules.

In one embodiment of the method, the biomolecules are separated into a phospholipid-containing region.

In another embodiment, the phospholipid-containing region is a raft phase or raft-like region, a 1-Palmitoyl-2-oleoyl-sn-glycero-3-phosphocholine(POPC)-rich region, a cholesterol (Chol) -rich region or a N-palmitoyl-D-erythro-sphingosylphosphorylcholine (PSM)-rich region.

A method for sorting biomolecules is also provided. In one embodiment, the method comprises the steps of:
introducing the biomolecules into a supported lipid bilayer;
patterning a supported lipid bilayer comprising a plurality of stable coexistent lipid phases;
applying an electric field or hydrodynamic flow to move the biomolecules through the supported lipid bilayer;
sorting migrating biomolecules based on their preference for heterogeneous regions in the supported lipid bilayer;
collecting sorted biomolecules in a quantification area; and classifying the sorted biomolecules based on their affinity for a particular lipid phase.

In another embodiment of the method, the biomolecules are sorted into a phospholipid-containing region.

In another embodiment of the method, the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

In another embodiment, the method comprises the step of determining the ratio of biomolecules collected in raft versus fluid phases. Determination of the ratio can be accomplished by techniques known in the art, for example, by mass spectrometry.

A method for assaying biomolecule partitioning preference is also provided. In one embodiment, the method comprises the steps of:
introducing biomolecules into a supported lipid bilayer;
patterning a supported lipid bilayer comprising a plurality of stable coexistent lipid phases;
applying an electric field or hydrodynamic flow to move the biomolecules through the supported lipid bilayer;
determining the partitioning of the migrating biomolecules based on their preference for heterogeneous regions in the supported lipid bilayer over time.

In one embodiment of the method, the partitioning preference of the biomolecule (e.g., cell membrane species) is a preference for partitioning into a raft-like region.

In another embodiment of the method, the biomolecules are sorted into a phospholipid-containing region.

In another embodiment of the method, The method of claim 17 wherein the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

A method for assaying interaction of a first biomolecule with a second biomolecule is also provided. In one embodiment, the method comprises the steps of:
providing a microfluidic channel;
providing a supported lipid bilayer comprising a plurality of stable coexistent lipid phases, wherein the supported lipid bilayer is patterned within the microfluidic channel;
introducing the first cell membrane species into the first phase of two phases of the supported lipid bilayer; and
applying a stimulus to induce mixing and/or biomolecule partitioning preference in the first membrane species positioned in the first phase such that interactions take place with the second cell membrane species positioned in the second phase.

In one embodiment of the method, the first or the second biomolecule is sorted into a phospholipid-containing region. In another embodiment of the method, three or more biomolecules can be sorted.

In another embodiment of the method, the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

In another embodiment of the method, the stimulus is (or results from) binding of a small molecule, a pH change, a temperature change, an ionic strength change, a chemical stimulus or an electrical stimulus.

In another embodiment, the method comprises the step of monitoring or determining a change in location of the first or second biomolecule.

In another embodiment, the method comprises the step of monitoring or determining a change in activity of the first or second biomolecule.

In another embodiment, the method comprises the step of monitoring or determining a change in function of the first or second biomolecule. Subsequent changes in activity as a result of such interactions can also be monitored or determined.

For any of the above-described methods, the biomolecule(s) can be cell membrane species.

For any of the above-described methods, the plurality of stable coexistent lipid phases can be patterned within the microfluidic channel.

For any of the above-described methods, the supported lipid bilayer can comprise at least two different stable, coexistent lipid phases in controllable or preselected spatial or temporal geometries.

For any of the above-described methods, the method can comprise the step of applying an electric field or fluidic flow to the biomolecules.

In another embodiment, a method for scaling up or testing a plurality of membranes phases or compositions comprising repeating patterning of the two phases to extend throughput for separation and sorting applications is also provided. In one embodiment, the scale-up can consist of multiple different phases within a device, rather than only two phases, whereby the partitioning or other interactions of biomolecules of interest can be assayed.

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated or enlarged to facilitate an understanding of the invention.

FIG. 1. An example of the function of cell membrane heterogeneity. Glycolipids initially sequestered in a microdomain can be released to surrounding lipid phase and then interact with membrane proteins within that phase. Interactions between glycolipid and proteins can induce activity changes in proteins.

FIGS. 2a-c. Formation of patterned phase coexistent supported lipid bilayers. (a) The formation of a solid-supported lipid bilayer via vesicle fusion. (b) The tertiary phase diagram of cholesterol (Chol), N-palmitoyl-D-erythro-sphingosylphosphorylcholinesphingomyelin (PSM, also referred to herein as “SM”), and palmitoyl-oleoyl-phosphatidylcholine (POPC) at 23° C. adapted from Veatch et al. (Veatch, S. L.; Keller, S. L. Physical Review Letters 2005, 94, 148101). The gray region is the two-phase coexistent region. The dark line is a hypothetical tie line. The positions where the tie line crosses the boundary of the gray region indicates the phase coexistent compositions, denoted as “F” and “R” for fluid and raft phases respectively. A conventional model system approach generates a two-phase coexistent region by preparing a composition in the gray region and letting the system itself phase separate, causing difficulties in controlling phase domain size and location. Our approach is to separately prepare the lipid vesicles with F and R compositions and pattern the two phases to the desired size and location. (c) Various patterning techniques can be used to pattern the two phases. Two examples are shown here: PDMS stamping or laminar flow in a microfluidic device. Upper image: a fluorescence image of stamped bilayers with two stable phases. The circle region contains a raft composition (60 mol % PSM, 40 mol % Chol) with no fluorophore. The region outside circles contains a fluid phase bilayer (70 mol % POPC, 20 mol % PSM, 10 mol % Chol) with 0.1 mol % fluorophore to aid visualization. Lower image: a fluorescence image of the microfluidic partitioning channel with two stable phases. The top half of the channel contains a raft composition (60 mol % PSM, 40 mol % Chol) with 0.1 mol % fluorophore to aid visualization. The bottom half of the channel contains a fluid phase bilayer (70 mol % POPC, 20 mol % PSM, 10 mol % Chol), with no fluorophore.

FIG. 3. Illustration of how patterned bilayers of a BPD can be made and used for separation purposes. In one embodiment of the method for making a BPD, the steps can comprise: (A) Form a bilayer via vesicle fusion of one composition, then (B) selectively remove by stamping regions where the second phase is desired. Back fill with the second vesicles (B). Stamp out a thin section of bilayer where a third vesicle formulation fuses; these vesicles contain the species to be separated (denoted by the mixture of light and dark circles arranged in a thin rectangle). Apply an electric field to induce the mixed charged species to migrate through the bilayer and partition as they sample the different regions of the bilayer. (3) Upper image: results of experiments showing the exclusion of BODIPY DHPE lipids from rafts regions (rafts are black circles containing no fluorophore). Lower image: results of experiments showing the preferential partitioning of GM1 lipids into raft regions. The GM1 is labeled with Alexa- 594, which makes it, and the rafts in which they are located, appear bright.

FIG. 4. Illustration of a BPD with three distinct bilayers: a loading bilayer with the proteins to sort (1-2), a raft membrane (3-5), and a fluid phase membrane (4-6). The raft and fluid phase membranes are healed to one another where they run parallel and meet in the main horizontal channel.

FIGS. 5a-f. An overview illustration of the microfluidic channel and the loading and patterning of bilayers into the device via vesicle fusion and laminar flow patterning. One side (lower side) represents the raft phase bilayer, the upper side is the POPC-rich phase (also called fluid phase) bilayer. The dotted region represents the load bilayer that is the same composition as the POPC-rich phase bilayer, except that it contains the biomolecules of interest to be analyzed or assayed. Lighter and darker circles represent BODIPY-DHPE (lighter) and head-labeled GM1 lipids (darker). The arrows show the direction of the flow and streamlines as the pattern is being formed and loaded.

FIG. 6. A schematic drawing of one embodiment of the BPD described herein.

FIG. 7. An illustration of an embodiment of a method for separating and sorting a biomolecule of interest using the BPD.

FIGS. 8a-e. Measuring partitioning kinetics in the BPD. (a) A bird's eye view fluorescent image of the microfluidic device and loading bilayer containing Alexa594-GM1 and head-labeled BODIPY-DHPE. The POPC-rich (fluid) phase is loaded in the top part of the main channel while the raft phase is in the bottom half. Both of these phases are initially devoid of any fluorophore; the interface between the phases and the boundary of the microchannel are marked by dashed lines that have been superimposed on the image. The white box in (a) is the control volume used for data analyses for kinetic measurements. (b) Hydrodynamic force induced by the bulk flow in the microchannel is applied to the direction denoted by the large white arrow to move the mixed species originating in the side channel down the POPC-rich-phase membrane into the main chamber. An increasing amount of GM I is extracted into the raft phase in time during the transport. This is indicated by the smaller white arrows. (c) Eventually the hydrodynamic flow is stopped and the system is allowed to equilibrate for 2 hrs. Most of GM1 partitions to the raft phase and most of BODIPY-DHPE remains in the POPC-rich phase. (d) A hydrodynamic flow is applied in the opposite direction to move the target species back down the channel. (e) is the corresponding amount of each species in the raft phase region in time. The upper line is GM1 and lower line in BODIPY-DHPE. See also FIGS. 12 and 13a.

FIG. 9a. Example of an embodiment of the BPD for high throughput strategy (or screening) or multiple composition assay.

FIG. 9b. Schematic drawing of the change in affinity of biomolecules before and after stimuli in an embodiment of the BPD.

FIG. 10. (Left) Dependence of protein activity on local lipid environment. (Right) Triggering the phase mixing or change in affinity of biomolecules for one phase can lead to different local lipid environment and subsequent change in activity. A 2-channel BPD can be used to investigate protein activity changes. Proteins initially sequestered outside the raft phase (bottom) can interact with molecules initially inside the raft phase after they are released from the raft phase upon triggered stimuli. When the interaction occurs, the protein becomes active as indicated by the change in color from dark to light.

FIG. 11. Illustration of a BPD designed to measure kinetics of membrane-bound biomolecule partitioning. Laminar flow in a microfluidic channel is used to create stripes of lipid raft phase and fluid phase in parallel along the main microchannel and to load a mixture of biomolecules to assay (darker and lighter) in the side channel. The interface between the phases is contiguous, allowing molecules to diffuse across this interface and partition (short arrow) into the raft phase as they are transported down the main channel by hydrodynamic force from bulk buffer flow through the channel (long arrow). Note that the membrane phases and loading bilayers are all adsorbed to the channel walls and the partitioning is taking place laterally within the two-dimensional plane of the bilayer.

FIGS. 12a-d. Left side: (a) A bird's eye view fluorescent image of the microfluidic device and loading bilayer containing Alexa594-G_{M1} and head-labeled BODIPY-DHPE. The POPC-rich phase is loaded in the top part of the main channel while the raft phase is in the bottom half. Both of these phases are initially devoid of any fluorophore; the interface between the phases and the boundary of the microchannel are marked by dashed lines that have been superimposed on the image. The white box in (a) is the control volume used for data analyses for kinetic measurements. The red color in the raft region close to the load shows that some of the head-labeled G_{M1} already started to partition into raft phase during the preparation step. (b) Hydrodynamic force induced by the bulk flow in the microchannel is applied to the direction denoted by the large white arrow to move the mixed species originating in the side channel down the POPC-rich-phase membrane into the main chamber. An increasing amount of G_{M1} is extracted into the raft phase in time during the transport. This is indicated by the smaller white arrows. (c) Eventually the hydrodynamic flow is stopped and the system is allowed to equilibrate for 2 hrs. Most of G_{M1} partitions to the raft phase and most of BODIPY-DHPE remains in the POPC-rich phase. (d) A hydrodynamic flow is applied in the opposite direction to move the target species back down the channel.

FIGS. 13a-c. Partitioning kinetic measurement of Alex594-head-labeled GM1 (top), BODIPY-tail-labeled GM1 (middle), and BODIPY-DHPE (bottom). (a) Species' content in the raft phase varying with time during the three stages: (1) flowing the species in the POPC-rich phase in contact with a fresh raft phase; (2) stopping the flow to let the system equilibrate; (3) applying reversed flow to allow the fresh POPC-rich phase entering into channel for dissociation step. (b) The corresponding images at the three different stages. (c) The chemical structures and labeling positions for (left to right) head-labeled G_{M1}, tail-labeled G_{M1}, and BODIPY-DHPE.

FIG. 14. An illustration of the patterned membrane in the control volume for the mass balance calculation used to determine the target species' association or dissociation kinetic rate with a specific membrane phase. In this case, the association and dissociation rates of G_{M1} and BODIPY-DHPE with the raft phase (lower) from the POPC-rich phase (upper) were examined. N_{R} is the amount of species in the raft phase region; L is the interface length between the raft phase and the POPC-rich phase F_{R,in} and F_{R,out} are the convective molar flow rates into and out the raft region. F_{F,in} and F_{F,out} are the convective molar flow rates into and out the POPC-rich region. w is the width of each phase. The subscripts “R” and “F” refer to the raft and POPC-rich phases, respectively.

FIG. 15. The amount of species in the raft phase region (N_{R}) as a function of a. In the legend, Alex594-head-labeled G_{M1} (steepest line)=HG_{M1}, BODIPY-tail-labeled G_{M1} (middle line)=TG_{M1}, and head-labeled BODIPY-DHPE (shallowest line)=H-BODIPY). The initial slopes (indicated by lines) are k₊ in Equation 3. The trend lines from the data of closed circles (1 mol % of the target species, flow rate=80 μl/min), open circles (0.75 mol % of target species, flow rate=80 μl/min), and stars (1 mol % of the target species, flow rate=40 μl/min) are similar, indicating the concentration profile does not influence the association rate constant and the association is first order in concentration. The lines deviate from linear behavior at later times because at later times the dissociation term and the molar flow rate term, shown in Equation 1, started to contribute to the accumulation amount in the raft phase (N_{R}) and can no longer be neglected.

FIG. 16. Plot of (dN_{R}(t)/dt)/L against C_{Ri}(t) to obtain the dissociation rate constant, k. The slope of the lines are the k. for each biomolecule. These values are listed in Table 1. H-BODIPY=head-labeled BODIPY-DHPE , TG_{M1}=tail-labeled G_{M1}, HG_{M1}=head-labeled G_{M1}.

FIGS. 17a-b. Stability of patterned membranes inside the microfluidic channel. (a) Two-phase coexistent compositions: the membrane in the top half of the channel (between dashed lines) contains 70/20/10 molar ratio of POPC/PSM/Chol doped with a mixture of 1 mol % BODIPY-DHPE and 1 mol % head-labeled G_{M1} (Alexa-594) at time=0. The membrane in the bottom half of the same channel contains 60/40 molar ratio of PSM/Chol with no fluorophores initially. The leftmost panel is the image taken 10 min after the membrane was prepared, after some partitioning has occurred between the phases. BODIPY-DHPE prefers the POPC-rich phase. After about four hours, the interface between the two phases based on the fluorescence intensity of BODIPY can still be clearly observed, indicating that the phase separation is stable. (b) POPC-rich phase composition only: the membrane in the top half of the channel contains 70/20/10 molar ratio of POPC/PSM/Chol doped with 1 mol % BODIPY-DHPE and 1 mol % head-labeled _{GMI} as in (a); however, the membrane in the bottom half contains the same membrane composition (70/20/10 molar ratio of POPC/PSM/Chol) with no fluorophores initially. At four hours after formation of the supported bilayers, no interface between the two regions can be observed (the bilayers became fully mixed). The entire channel width is 100 μm.

FIG. 18. Correlation between fluorescence intensity and concentration of fluorescent molecules in the range of the concentrations used in our experiments. Triangle data points correspond to fluorescence in raft phase; circles correspond to POPC-rich (fluid) phase.

FIG. 19. Illustration of how a target species' concentration (line) changes in the transitional interface region between the bulk raft phase (left) and bulk POPC-rich phase (right). The dotted lines indicate the boundaries of the interface region. F_{RT} is the net species passing rate from the bulk raft phase region to the transitional layer and F_{FT} is the net species passing rate from the bulk POPC-rich phase to the transitional layer. C_{Fi} is the species concentration at the interface boundary between the bulk POPC-rich phase and the transitional layer. C_{Ri} is the species concentration at the interface boundary between the bulk raft phase and the transitional layer.

FIGS. 20a-h. Bird's eye view fluorescent images and fluorescence intensity profile across the channel during the association stage, equilibrating stage and dissociation stage. (a) The image before any hydrodynamic force was applied to move head-labeled G_{M1} and head-labeled BODIPY-DHPE. The POPC-rich phase was loaded in the top part of the main channel while the raft phase was in the bottom half. Both of these phases were initially devoid of fluorophore; the interface boundaries between the phases and the boundary of the microchannel are marked by dashed lines that have been superimposed on the image. (b) Association stage: a hydrodynamic force was applied to the right to bring the target species into the main chamber. (c) Equilibrating stage: the hydrodynamic force was stopped and the system was allowed to sit for 2 hrs to equilibrate. (d) Dissociation stage: a reversed hydrodynamic flow was applied to move the target species away from the main chamber. (e) The final state after most of the species were removed from the sight of view. (f) Fluorescence intensity profile change with time along the line denoted in (a) during the association stage. The time interval between each profile was 2 min. (g) Intensity profile change during the equilibrating stage. The time interval was 20 min. (h) Intensity profile change during the dissociation stage. The time interval was 10 min.

FIGS. 21a-b. The concentration profile in the y direction (across the channel) during (a) the association stage and (b) the dissociation stage. The different lines indicate the concentration profile at a certain time. The right dashed line is the interface boundary of POPC-rich phase, and the left line is the interface boundary of raft phase, as defined according to the overall profile change with time. The region on the right is defined as the bulk POPC-rich phase; the region on the left is the bulk raft phase; and the region between the two dashed lines is the interface region. The dot denoted by C_{Fi}(tₙ) or C_{Ri}(tₙ) is the concentration at the interface boundary of either POPC-rich or raft phase at tₙ. The dashed region under the curve indicates the change of N_{R} between tₙ and tₙ₊₁.

FIG. 22a-b. (a) dN_{R}/dt from the slopes of N_{R}−t plot during the dissociation stage. (b) The C_{Ri} at the corresponding time can be found from the intensity profiles in the y direction (across the channel) during the dissociation stage.

FIG. 23. Plot of (dN_{R}(t)/dt)/L against C_{Ri}(t) to obtain dissociation rate constant, k.

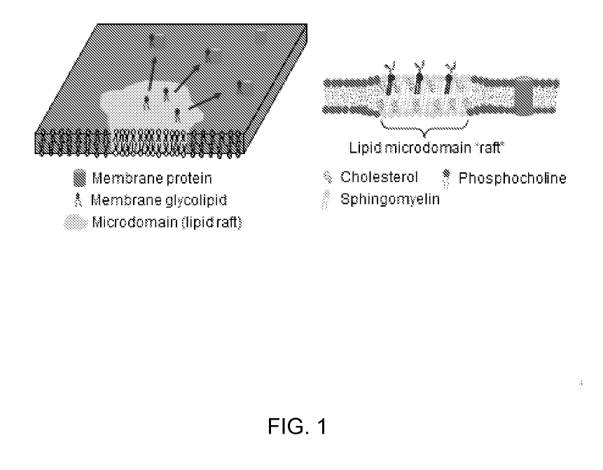

### 5. DETAILED DESCRIPTION OF THE INVENTION

A biomolecule partitioning device (BPD) is provided that can be used to separate and sort membrane species into raft-like membrane regions without using detergent or crosslinkers. The BPD can comprise one or more microfluidic channels coated with coexistent lipid phases (raft-like and fluid-like lipid compositions) as a contiguous supported lipid bilayer (SLB). The geometry of the phases can be patterned with spatial and temporal control within each channel. Methods for the separation and sorting are also provided. The method can comprise the steps of introducing cell membrane species into an SLB; patterning coexistent phases; applying an electric field or hydrodynamic flow to move the species; sorting migrating species into regions based on their partitioning preference; and collecting sorted species in a quantification area. The BPD can also be used to measure partitioning kinetics or to assay for activity changes of biomolecules as a function of local lipid environment.

In one embodiment, a biomolecule partitioning device (BPD) for partitioning biomolecules is provided. In one embodiment, the BPD comprises:
a substrate;
a microfluidic channel on the substrate;
a plurality of stable coexistent lipid phases, wherein the surface of the microfluidic channel is coated with a plurality of stable, coexistent lipid phases, thereby forming a supported lipid bilayer.

In another embodiment, the biomolecules are cell membrane species. The cell membrane species can be a protein, a glycolipid, a proteolipid, a proteoglycan or any other membrane species known in the art.

In another embodiment, the plurality of stable coexistent lipid phases comprises a raft-like or a fluid-like lipid composition. Once in the membrane, the biomolecules can partition into either phase.

In another embodiment, the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region. The phospholipid-containing region can contain any phospholipid or phospholipid derivative known in the art (e.g.,a phosphatidylcholine (or derivative thereof)). In another embodiment, the region can contain any cholesterol derivative known in the art.

In another embodiment, the plurality of stable coexistent lipid phases are patterned within the microfluidic channel.

In another embodiment, the BPD can comprise a cushion for reducing strong protein interaction with the surface of the microfluidic channel.

In another embodiment, the supported lipid bilayer comprises at least two different stable, coexistent lipid phases in controllable or preselected spatial or temporal geometries.

In another embodiment, an electric field or fluidic flow is applied to the biomolecule.

In another embodiment, the BPD comprises one or more collection channels or ports. In another embodiment, the BPD can comprise one or more loading channels or ports.

In another embodiment, the BPD can comprise two contiguous phases (e.g., FIG. 3) or multiple contiguous phases (e.g., FIG. 7). In other embodiments, the number of phases is 2-10, 10-20, 20-30, 30-40, 40-50, 50-60, 60-70, 70-80, 80-90, 90-100 or greater than 100. The multiple phases can all be unique (e.g., a, b, c, d, e . . . ), alternating repeating (e.g., a, b, a, b, . . . ) or other (e.g., a, b, a, c, a, d . . . or a, b, c, a, d, e . . . ). Such permutations and variations will be apparent to the skilled artisan.

A method for separating biomolecules is also provided. In one embodiment, the method comprises the steps of:
providing a microfluidic channel;
providing a supported lipid bilayer comprising a plurality of stable coexistent lipid phases, wherein the supported lipid bilayer is patterned within the microfluidic channel;
introducing the biomolecules into the supported lipid bilayer;
applying an electric field or hydrodynamic flow to move the biomolecules through the supported lipid bilayer;
separating migrating biomolecules based on their preference for heterogeneous regions in the supported lipid bilayer; and
collecting the separated biomolecules.

In one embodiment of the method, the biomolecules are separated into a phospholipid-containing region.

In another embodiment, the phospholipid-containing region is a raft phase or raft-like region, a 1-Palmitoyl-2-oleoyl-sn-glycero-3-phosphocholine(POPC)-rich region, a cholesterol (Chol) -rich region or a N-palmitoyl-D-erythro-sphingosylphosphorylcholine (PSM)-rich region.

A method for sorting biomolecules is also provided. In one embodiment, the method comprises the steps of:
introducing the biomolecules into a supported lipid bilayer;
patterning a supported lipid bilayer comprising a plurality of stable coexistent lipid phases;
applying an electric field or hydrodynamic flow to move the biomolecules through the supported lipid bilayer;
sorting migrating biomolecules based on their preference for heterogeneous regions in the supported lipid bilayer;
collecting sorted biomolecules in a quantification area; and
classifying the sorted biomolecules based on their affinity for a particular lipid phase.

In another embodiment of the method, the biomolecules are sorted into a phospholipid-containing region.

In another embodiment of the method, the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

In another embodiment, the method comprises the step of determining the ratio of biomolecules collected in raft versus fluid phases. Determination of the ratio can be accomplished by techniques known in the art, for example, by mass spectrometry.

A method for assaying biomolecule partitioning preference is also provided. In one embodiment, the method comprises the steps of:
introducing biomolecules into a supported lipid bilayer;
patterning a supported lipid bilayer comprising a plurality of stable coexistent lipid phases;
applying an electric field or hydrodynamic flow to move the biomolecules through the supported lipid bilayer;
determining the partitioning of the migrating biomolecules based on their preference for heterogeneous regions in the supported lipid bilayer over time.

In one embodiment of the method, the partitioning preference of the biomolecule (e.g., cell membrane species) is a preference for partitioning into a raft-like region.

In another embodiment of the method, the biomolecules are sorted into a phospholipid-containing region.

In another embodiment of the method, The method of claim 17 wherein the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

A method for assaying interaction of a first biomolecule with a second biomolecule is also provided. In one embodiment, the method comprises the steps of:
providing a microfluidic channel;
providing a supported lipid bilayer comprising a plurality of stable coexistent lipid phases, wherein the supported lipid bilayer is patterned within the microfluidic channel;
introducing the first cell membrane species into the first phase of two phases of the supported lipid bilayer; and
applying a stimulus to induce mixing and/or biomolecule partitioning preference in the first membrane species positioned in the first phase such that interactions take place with the second cell membrane species positioned in the second phase.

In one embodiment of the method, the first or the second biomolecule is sorted into a phospholipid-containing region. In another embodiment of the method, three or more biomolecules can be sorted.

In another embodiment of the method, the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

In another embodiment of the method, the stimulus is (or results from) binding of a small molecule, a pH change, a temperature change, an ionic strength change, a chemical stimulus or an electrical stimulus.

In another embodiment, the method comprises the step of monitoring or determining a change in location of the first or second biomolecule.

In another embodiment, the method comprises the step of monitoring or determining a change in activity of the first or second biomolecule.

In another embodiment, the method comprises the step of monitoring or determining a change in function of the first or second biomolecule. Subsequent changes in activity as a result of such interactions can also be monitored or determined.

For any of the above-described methods, the biomolecule(s) can be cell membrane species.

For any of the above-described methods, the plurality of stable coexistent lipid phases can be patterned within the microfluidic channel.

For any of the above-described methods, the supported lipid bilayer can comprise at least two different stable, coexistent lipid phases in controllable or preselected spatial or temporal geometries.

For any of the above-described methods, the method can comprise the step of applying an electric field or fluidic flow to the biomolecules.

In another embodiment, a method for scaling up or testing a plurality of membranes phases or compositions comprising repeating patterning of the two phases to extend throughput for separation and sorting applications is also provided. In one embodiment, the scale-up can consist of multiple different phases within a device, rather than only two phases, whereby the partitioning or other interactions of biomolecules of interest can be assayed.

Classification of membrane biomolecules using the BPD and BPD-based assay methods can greatly increase understanding of cell membrane organization and function, and aid in understanding how malfunctions lead to disease as well as use as a diagnostic test for disease. In addition, the BPD can quantify shifts in partitioning behavior of biomolecules as a function of membrane composition, activation level, and exposure to small molecules. This latter feature can be employed in biosensor devices, assay platforms for proteomic research, or screening tools for pharmaceuticals.

For clarity of disclosure, and not by way of limitation, the detailed description of the invention is divided into the subsections set forth below.

### 5.1. Biomolecule Partitioning Device (BPD) for Sorting, Classifying, and Assaying Partition Behavior of Cell Membrane Biomolecules

A platform for studying membrane proteins, lipids, and other biomolecules is provided that not only preserves the membrane environment but also can be tuned for a host of advantageous reasons. The platform (or device), referred to herein as a biomolecule partitioning device (BPD) can be used to assay protein, glycolipid, proteolipid, etc., partitioning behavior and how it changes based on interactions with other species.

The BPD and assays based thereon fill an unmet need in the research marketplace. The device can classify the partitioning behaviors of proteins without the use of detergent and thus can shed light on the long-standing raft controversy and aid in membrane proteomic analysis. The controversy over detergents makes it impossible to interpret detergent-driven data. The device disclosed herein bypasses this issue by sorting proteins based on their preferences for association with different lipids, which can be collected and analyzed later, as will be described below.

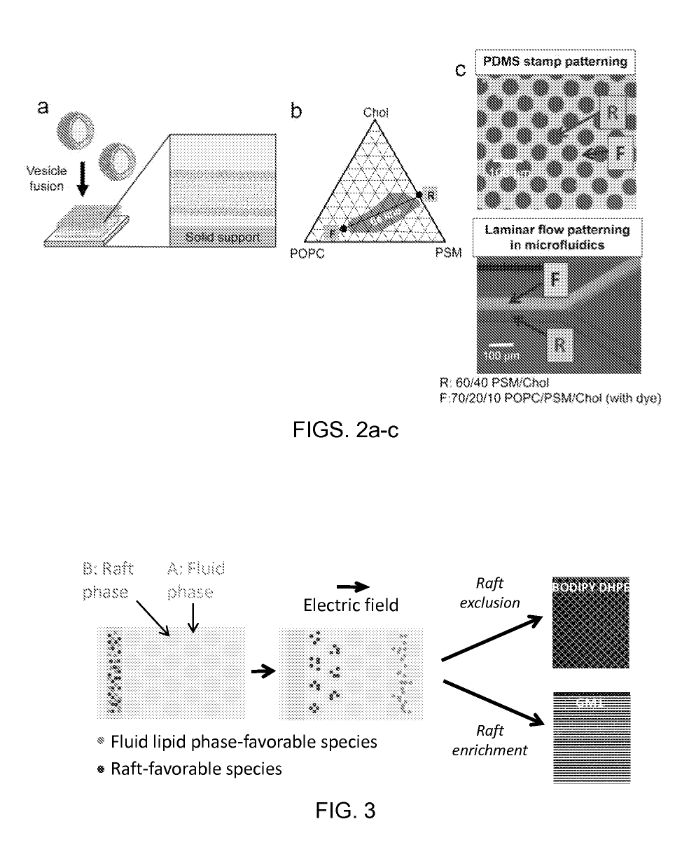

In one embodiment, the BPD comprises a substrate. A microfluidic channel is positioned on the substrate and the surface of the substrate is coated with supported lipid bilayers (SLB) (a membrane mimic to house the proteins or other species). A cushion can be employed, using methods known in the art, to reduce strong protein interaction with the support. The SLB is one component material which makes this proposed assay platform possible (FIG. 2a). SLBs are excellent mimics of the cell membrane because they maintain two-dimensional fluidity of the constituents, proper orientation, and maintenance of hydrophobic cores of membrane species, just like in real cells. Because of these features, species such as proteins can be transported by electrophoresis or hydrodynamic flow while suspended in SLBs. Electrophoresis can be used to transport charged species, while hydrodynamic flow can be used for both uncharged and charged species. The BPD sorts migrating proteins based on their preference for heterogeneous regions in the bilayer that can be created and patterned as desired by the user, so that sorted proteins can be collected in a designated area for quantification.

In one embodiment, the separation bilayer is patterned with multiple phases (raft and non-raft) that interact with species differentially to effect a separation analogous to a chromatographic separation strategy (FIG. 3). Here differently partitioning behavior serves to change the mobility of one species relative to the other such that they become separated. FIG. 3 illustrates how patterned bilayers of a BPD can be made and used for separation purposes. In one embodiment of the method for making a BPD, the steps can comprise: (A) Forming a bilayer via vesicle fusion of one composition, then (B) selectively remove by stamping regions where the second phase is desired. Back filling with the second vesicles (B). Stamping out a thin section of bilayer where a third vesicle formulation fuses; these vesicles contain the species to be separated (denoted by the mixture of light and dark circles arranged in a thin rectangle). An electric field can then be applied to induce the mixed charged species to migrate through the bilayer and partition as they sample the different regions of the bilayer. (3) Upper image shows results of experiments showing the exclusion of BODIPY DHPE lipids from rafts regions (rafts are black circles containing no fluorophore). Lower image shows results of experiments showing the preferential partitioning of GM1 lipids into raft regions. The GM1 is labeled with Alexa-594, which makes it, and the rafts in which they are located, appear bright.

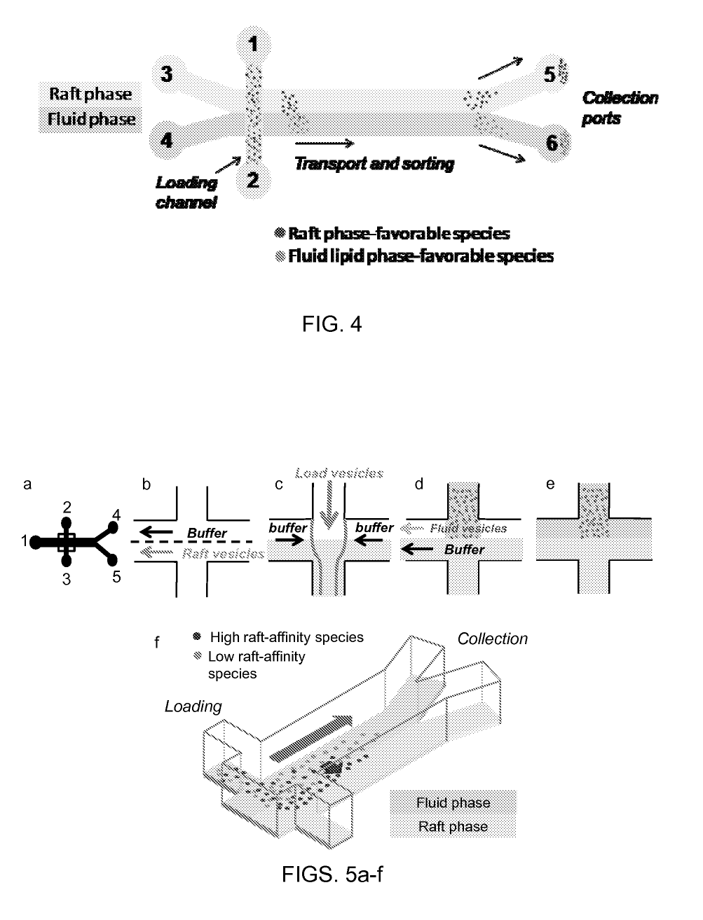

A second embodiment of the device is shown in FIG. 4. FIG. 4 shows a bird's eye view of an embodiment of a BPD that is a protein sorting device: a loading bilayer with the proteins to sort (1-2), a raft membrane (3-5), and a fluid phase membrane (4-6). The raft and fluid phase membranes are healed to one another where they run parallel and meet in the main horizontal channel.

In FIG. 4, the geometry of the raft phase runs parallel with the fluid phase to create an extraction geometry. Molecules loaded into the device (channel 1-2) are charged and are pulled down the main horizontal channel towards 5 and 6 ports. As the species migrate, they can sample by diffusion across the main channel in the membrane and partition into the preferred phase. In this embodiment, darker species prefer the raft phase and so enrich there, while the lighter species prefers the fluid phase and enrich there. At the exit ports of the device, molecules can be collected for later analysis by proteomic techniques such as mass spectrometry. The proteins can then be obtained from ports 5 and 6 at the end of the run or collected as fractions during the run and analyzed, such as by mass spectrometry. In other embodiments, ports 3 and 4 may be combined into one port.

### 5.2. Methods for Making and Using the Biomolecule Partitioning Device (BPD)

A method for making the BPD is also provided. FIGS. 2a-c show formation of patterned phase coexistent supported lipid bilayers in one embodiment of the method. FIG. 2a shows the formation of a solid-supported lipid bilayer via vesicle fusion. FIG. 2b shows the tertiary phase diagram of cholesterol (Chol), N-palmitoyl-D-erythro-sphingosylphosphorylcholine (PSM), and palmitoyl-oleoyl-phosphatidylcholine (POPC) at 23° C. adapted from Veatch et al. (Veatch, S. L.; Keller, S. L. Physical Review Letters 2005, 94, 148101). The gray region is the two-phase coexistent region. The dark line is a hypothetical tie line. The positions where the tie line crosses the boundary of the gray region indicates the phase coexistent compositions, denoted as “F” and “R” for fluid and raft phases respectively. A conventional model system approach generates a two-phase coexistent region by preparing a composition in the gray region and letting the system itself phase separate, causing difficulties in controlling phase domain size and location. In one embodiment of the method, lipid vesicles are separately prepared with two compositions, e.g., with F and R compositions, and the two phases are patterned to the desired size and location. FIG. 2c shows that various patterning techniques known in the art dan be used to pattern the two phases. Two examples are shown in FIG. 2c: PDMS stamping or laminar flow in a microfluidic device. Upper image: a fluorescence image of stamped bilayers with two stable phases. The circle region contains a raft composition (60 mol % PSM, 40 mol % Chol) with no fluorophore. The region outside circles contains a fluid phase bilayer (70 mol % POPC, 20 mol % PSM, 10 mol % Chol) with 0.1 mol % fluorophore to aid visualization. Lower image: a fluorescence image of the microfluidic partitioning channel with two stable phases. The top half of the channel contains a raft composition (60 mol % PSM, 40 mol % Chol) with 0.1 mol % fluorophore to aid visualization. The bottom half of the channel contains a fluid phase bilayer (70 mol % POPC, 20 mol % PSM, 10 mol % Chol), with no fluorophore.

FIGS. 5a-f shows one embodiment of a method for constructing the BPD. Lipids are used to demonstrate sorting in this embodiment of the PBD. GM₁ (darker), which is a known raft resident and BODIPY DHPE (lighter) lipid, which is known to be excluded from rafts are used as exemplary biomolecules to demonstrate sorting. The load channel of a mixture of G_{M1} and BODIPY DHPE is formed by vesicle fusion in the channel of FIG. 5. Buffer flow prevents these vesicles from entering the other channels where the extraction membranes will be formed. Once the load membrane is formed, the two compositions of vesicles are flowed, one of raft composition and one of fluid lipid phase, from the channel. Because the flow is laminar in the microfluidic device, the vesicles to not mix, but rupture to form the two adjacent bilayers, as can be seen in FIG. 2c (for representation purposes, the raft phase was explicitly fluorescently labeled with 0.1 mol % fluorophore to show the well-defined interface with an unlabeled fluid lipid phase). The top half of the channel contains the raft composition with 0.1 mol % fluorophore to visualize it with the fluorescent microscope. The bottom half of the same channel contains another bilayer of different composition which has no fluorophore. When in actual use, the raft phase is devoid of fluorophore. Thus, it is clear to visualize the well-defined dividing line down the middle of the channel where the two bilayers meet. The two phases are stable, as described in more detail below in Section 6.1 “Supplementary Materials and Methods.

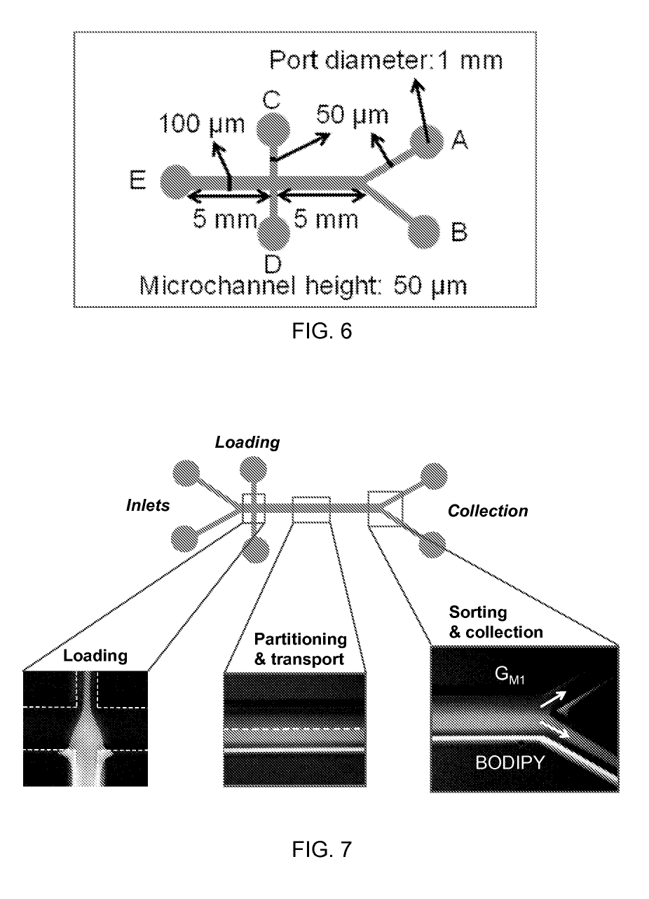

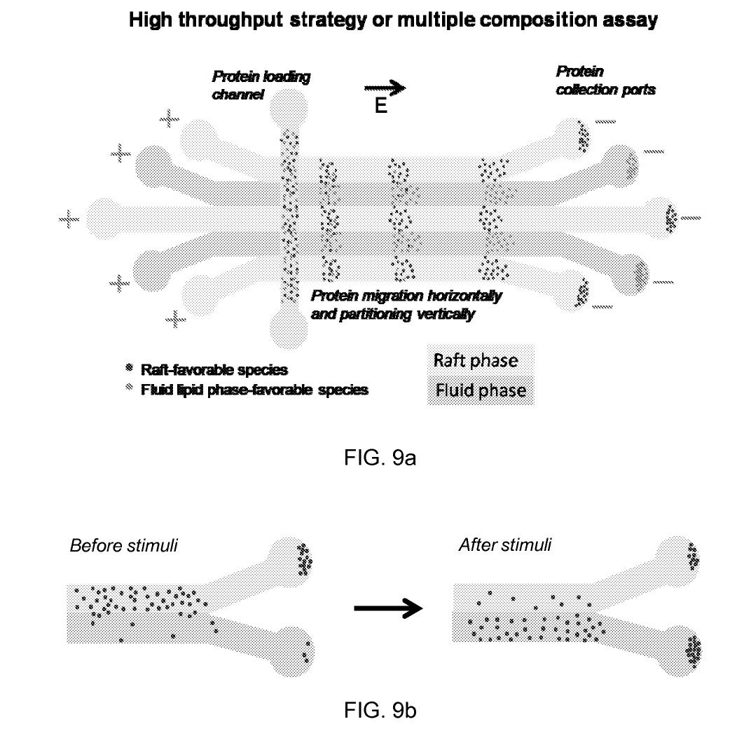

FIG. 6 is a schematic diagram of another embodiment of the BPD. In other embodiments, a parallel periodic array of membrane compositions can be created within one device, as in FIG. 9a. As show in the embodiment of FIG. 6, the microchannel height is 50 μm, with a channel length from inlet (C-D) to where the channels for port A and B diverge of 5 mm, a main channel width of 100 μm, port channel width of 50 μm and a port diameter of 1 mm. In other embodiments, the microchannel height can range from 10-200 μm, the width from 10 -500 μm, the length from 1-25 mm and the port diameter from 10 μm to 5 mm. Other suitable dimensions will be readily apparent to the skilled artisan.

FIG. 7 shows an embodiment of the BPD and how the species can be transported down the channel in this embodiment. The enrichment in time becomes apparent with Alexa-labeled GM1 accumulating in the raft phase (top) and BODIPY DHPE enriching in the fluid phase (bottom).

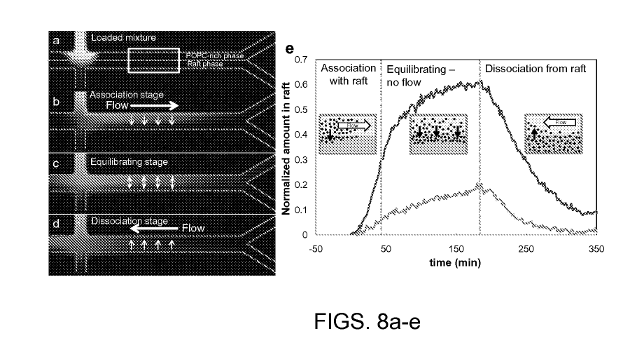

FIGS. 8a-e shows an example of measuring partitioning kinetics in one embodiment of the BPD. In FIGS. 8a-e, the time course of enrichment of G_{M1} in the raft phase (bottom half of the channel) and enrichment of BODIPY DHPE lipid in fluid phase in upper half of the microfluidic channel is shown. FIG. 8a. A bird's eye view fluorescent image of the microfluidic device and loading bilayer containing Alexa594-GM1 and head-labeled BODIPY-DHPE. The POPC-rich (fluid) phase is loaded in the top part of the main channel while the raft phase is in the bottom half. Both of these phases are initially devoid of any fluorophore; the interface between the phases and the boundary of the microchannel are marked by dashed lines that have been superimposed on the image. The white box in FIG. 8a is the control volume used for data analyses for kinetic measurements. FIG. 8b. Hydrodynamic force induced by the bulk flow in the microchannel is applied to the direction denoted by the large white arrow to move the mixed species originating in the side channel down the POPC-rich-phase membrane into the main chamber. An increasing amount of GM1 is extracted into the raft phase in time during the transport. This is indicated by the smaller white arrows. FIG. 8c. Eventually the hydrodynamic flow is stopped and the system is allowed to equilibrate for 2 hrs. Most of GM1 partitions to the raft phase and most of BODIPY-DHPE remains in the POPC-rich phase. FIG. 8d. A hydrodynamic flow is applied in the opposite direction to move the target species back down the channel. FIG. 8e is the corresponding amount of each species in the raft phase region in time. The upper line is GM1 and lower line in BODIPY-DHPE.

In another embodiment, multiple phases within the channel can be created that represent different membrane configurations. This can be used in a high throughput screening (HTS) format to analyze different treatment scenarios.

In one embodiment, the BPD comprises a supported, planar bilayer. This can be used to mimic the cell membrane which is amenable to many downstream tools for later biophysical assay including various microscopy tools. This configuration is also compatible with collection of sorted species to facilitate further identification of species or studies using other tools such as mass spectrometry or proteomic tools.

In another embodiment, the membrane can be patterned in a reproducible, predictable manner using methods known in the art. This can be used for sorting and further studies of the molecules without having to additionally label the membrane to distinguish regions of interest.

The art known properties of microfluidic devices can be used to aid in fluid management and BPD design.

An advantageous aspect of the BPD is that detergent or crosslinking agents do not need to be used.

FIG. 9a shows an embodiment in which the device can be extended for high throughput screening by creating periodic microfluidic channels as shown. This plurality of microfluidic channels can be alternate raft/fluid phase for increasing the amount of extraction of each species (as shown) or each channel can be designed for phase separated regions with different compositions and/or components to determine preference of a species with a certain composition of raft.

FIG. 9b shows a schematic drawing of the change in affinity of biomolecules before and after stimuli in an embodiment of the BPD.

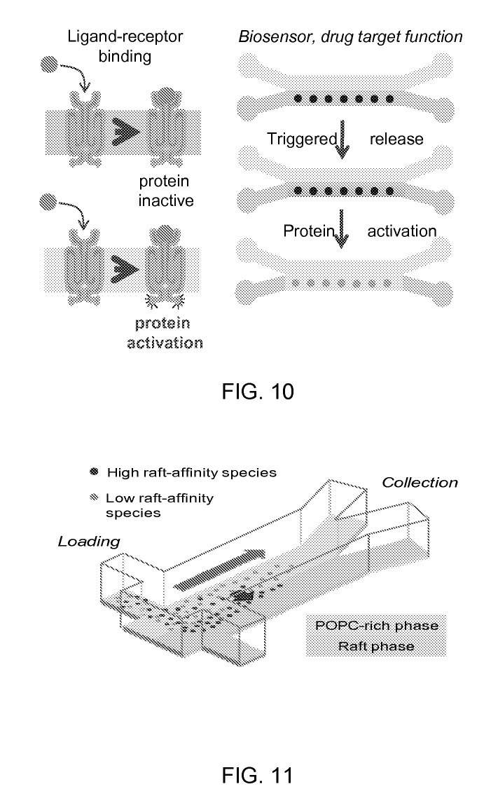

In another embodiment, the BPD can function as a sensor or detector for detecting a change in state of a biomolecule (see, e.g., FIG. 10). For example, the activation, conformational change, change in binding affinity or other change of state known in the art for a biomolecule can be detected by observing whether the biomolecule ‘migrates’ from one phase to another. FIG. 10 shows an embodiment of the BPD that functions as a sensor. On the left, dependence of protein activity on local lipid environment is shown. At the right, triggering the phase mixing or change in affinity of biomolecules for one phase can lead to different local lipid environment and subsequent change in activity. In a specific embodiment, a multi-channel BPD (e.g., 2 channels) can be used to investigate protein activity changes. Proteins initially sequestered outside the raft phase (bottom) can interact with molecules initially inside the raft phase after they are released from the raft phase upon triggered stimuli. When the interaction occurs, the protein becomes active as indicated by the change in color from dark to light.

FIG. 11. Illustration of a BPD designed to measure kinetics of membrane-bound biomolecule partitioning. Laminar flow in a microfluidic channel is used to create stripes of lipid raft phase and fluid phase in parallel along the main microchannel and to load a mixture of biomolecules to assay (darker and lighter) in the side channel. The interface between the phases is contiguous, allowing molecules to diffuse across this interface and partition (short arrow) into the raft phase as they are transported down the main channel by hydrodynamic force from bulk buffer flow through the channel (long arrow). Note that the membrane phases and loading bilayers are all adsorbed to the channel walls and the partitioning is taking place laterally within the two-dimensional plane of the bilayer.

### 5.3. Uses of the Biomolecule Partitioning Device (BPD)

The BPD can be used to sort species and classify them as raft or non-raft residents to define the raft proteome.

The BPD can be used as a diagnostic tool for disease to determine if certain subpopulations of species from a patient sample are located in proper regions of the cell membrane.

The BPD can be used as a screening tool for drug interactions that modify partitioning behavior of certain species to change its regulatory effects for disease mitigation. The drug or potential drug compound can be added to the membrane sample prior to addition to the loading channel or it can be introduced containing the raft/fluid phase membranes to determine the effect during protein migration.

The BPD can be used to study the partition kinetics when a molecule is stimulated (FIG. 9b). The BPD can be used to monitor the change in partition behavior of species when subjected to stimuli, including but not limited to: temperature, enzyme interaction, soluble protein interactions, chemical stimuli, changes in buffer conditions (ionic strength, pH, etc.).

The BPD can be used to assay activity of proteins or other species in the membrane depending on the local lipid environment or interaction with specific membrane species (FIG. 10).

The BPD can be used to measure kinetics of membrane-bound biomolecule partitioning (FIG. 11). Laminar flow in a microfluidic channel is used to create stripes of lipid raft phase and fluid phase in parallel along the main microchannel and to load a mixture of biomolecules to assay (darker and lighter) in the side channel. The interface between the phases is contiguous, allowing molecules to diffuse across this interface and partition (short arrow) into the raft phase as they are transported down the main channel by hydrodynamic force from bulk buffer flow through the channel (long arrow). Note that the membrane phases and loading bilayers are all adsorbed to the channel walls and the partitioning is taking place laterally within the two-dimensional plane of the bilayer.

The BPD can be used as a tool to quantify protein-raft associations and changes in partition behavior as conditions change, without ever having to remove the protein from a membrane or subjecting it to detergent/denaturation treatments. For example, this device could be used to verify hypotheses that proteins change partition behavior after interaction or stimulation with small soluble molecules, such as therapeutic proteins or drug targets. Assays employing the device can quantify these changes by flowing the test molecules through the microfluidic channel and observing the change in protein partitioning. The BPD can sort membrane proteins based on preferences for lipid associations, while in a native-like membrane environment. Shifts in partitioning behavior of native proteins as a function of membrane composition, activation level, and exposure to small molecules, which is not possible in any in vitro assay platform available in the art. This information is vital to the pharmaceutical and therapeutic protein industries who design small molecules to interact with membrane species to target diseases such as hyperlipidemia, diabetes, and age-related diseases, where a change in the cell membrane is known to contribute to the disease manifestation. Knowing how the protein function depends on the local membrane and how that environment impacts the protein-therapeutic interaction is vital to assessing the efficacy of the medicine to mitigate disease and in designing better targets.

In sum, the BPD can handle membrane species in a native-like membrane environment to minimize loss of structure. It can be used to separate, sort, and collect membrane species using partitioning in heterogeneous membranes. It can also be used to classify the residency of glycolipids in specific kinds of membrane compositions near physiological conditions, i.e., the extraction efficiency depends on the chemical structure. The BPD can also be used to assess partitioning kinetics of glycolipids to the raft phase within a heterogeneous membrane. The BPD can also be used to interrogate protein-lipid interactions and the dependence of protein function on specific lipid microenvironments.

The following examples are offered by way of illustration and not by way of limitation.

### 6. EXAMPLES

### 6.1. Example 1

### Measuring the Partitioning Kinetics of Membrane Biomolecules Using Patterned Two-Phase Coexistent Lipid Bilayers

### 6.1.1. Introduction

This example discloses a method for measuring the partitioning kinetics of membrane biomolecules to different lipid phases using a patterned supported lipid bilayer (SLB) platform composed of liquid-ordered (lipid raft) and liquid-disordered (unsaturated lipid-rich) coexistent phases. This approach removes the challenges in measuring partitioning kinetics using current in vitro methods due to their lack of spatial and temporal control of where phase separation occurs and when target biomolecules meet those phases. The laminar flow configuration inside a microfluidic channel allows SLBs to be patterned with coexistent phases in predetermined locations and thus eliminates the need for additional components to label the phases. Using a hydrodynamic force provided by the bulk flow in the microchannel, target membrane-bound species to be assayed can be transported in the bilayers. The pre-defined location of stably coexistent phases, in addition to the controllable movement of the target species allows the user to control and monitor when and where the target molecules approach or leave different lipid phases. Using this approach with appropriate experimental designs, the association and dissociation kinetic parameters were obtained for several membrane-bound species, including the glycolipid, G_{M1}, an important cell signaling molecule. Two different versions of G_{M1} were examined. Structural differences between them impacted the kinetics of association of these molecules to raft-like phases. This method can be extended to measuring the partitioning kinetics of other glycolipids, lipid-linked proteins, or transmembrane proteins with posttranslational modifications to provide insight into how structural factors, membrane compositions, and environmental factors influence dynamic partitioning.

Current understanding of the cell membrane suggests that it is a patchwork structure composed of multiple proteins and lipids that are not all freely diffusing and well-mixed, but rather can take part in dynamic microdomains (Edidin, M. Nature 2005, 4, 414; Simons, K.; Ikonen, E. Nature 1997, 387, 569; Sprenger, R. R.; Horrevoets, J. G. Proteomics 2007, 7, 2895; Shaw, A. S. Nature Immunology 2006, 7, 1139; Zheng, Y. Z.; Foster, L. J. Proteomics 2009, 72, 12; Simons, K.; Vaz, W. L. C. Annu. Rev. Biophys. Biomol. Struct. 2004, 33, 269; Pike, L. J. Journal of Lipid Research 2009, 50, S323; Lingwood, D.; Simons, K. Science 2010, 327, 46). Separation of distinct lipid membrane domains within the cell membrane has been suggested to play important roles in many cellular processes by providing various microenvironments to cluster or to isolate membrane biomolecules (Lingwood, D.; Simons, K. Science 2010, 327, 46; Simons, K.; Ikonen, E. Nature (London) 1997, 387, 569; Simons, K.; Toomre, D. Nature Reviews Molecular Cell Biology 2000, 1, 31). One class of liquid-ordered membrane domain, enriched in sphingolipids and cholesterol, is called a lipid raft (Simons, K.; Ikonen, E. Nature (London) 1997, 387, 569; Simons, K.; Toomre, D. Nature Reviews Molecular Cell Biology 2000, 1, 31; Pike, L. J. Journal of Lipid Research 2006, 47, 1597; Jacobson, K.; Mouritsen, O. G.; Anderson, R. G. W. Nat Cell Biol 2007, 9, 7). Under specific conditions, certain glycolipids, proteins, and other membrane species exhibit a high affinity for lipid rafts; while other species prefer the more disordered phase surrounding the rafts (Pike, L. J. Journal of Lipid Research 2009, 50, S323; Dietrich, C.; Volovyk, Z. N.; Levi, M.; Thompson, N. L.; Jacobson, K. Proceedings of the National Academy of Sciences 2001, 98, 10642; Kenworthy, A. K.; Nichols, B. J.; Remmert, C. L.; Hendrix, G. M.; Kumar, M.; Zimmerberg, J.; Lippincott-Schwartz, J. The Journal of Cell Biology 2004, 165, 735).

Identification of critical lipid-protein interactions and changes in affinity of certain biomolecules with lipid raft phases may be critical to understanding the causes of a number of diseases, including infertility (Selvaraj, V.; Bunke, D. E.; Asano, A.; Mcelwee, J. L.; Wolff, C. A.; Nelson, J. L.; Klaus, A. V.; Hunnicutt, G. R.; Travis, A. J. Journal of Andrology 2007, 28, 588), viral infection and associated diseases (Chazal, N.; Gerlier, D. Microbiol. Mol. Biol. Rev. 2003, 67, 226), Alzheimer's disease (Cordy, J. M.; Hooper, N. M.; Turner, A. J. Molecular Membrane Biology 2006, 23, 111) and other age-related diseases (Ohno-Iwashita, Y.; Shimada, Y.; Hayashi, M.; Inomata, M. Geriatr. Gerontol. Int. 2010, 10, S41; Fulop, T.; Larbi, A.; Dupuis, G.; Pawelec, G. Arthritis Research & Therapy, 5, 290), and therefore vital for designing the most effective therapeutic drugs to combat these diseases.

There are presently only limited approaches to identify and classify the affinity of membrane biomolecules to different lipid domains. Current in vivo methods attempt to characterize the residency of membrane molecules' to various lipid phases using detergent and/or high salt/alkaline pH to isolate insoluble membrane fractions and then correlate the contents of those fractions (Sprenger, R. R.; Horrevoets, J. G. Proteomics 2007, 7, 2895; Shaw, A. S. Nature Immunology 2006, 7, 1139). However, these assorted chemicals and conditions lead to variations in compositions from experiment to experiment and often contamination with species from other cellular compartments, which compromises the reliability of this approach.

Other strategies to identify microdomain residents involve the direct labeling of the intact cell membrane (Zheng, Y. Z.; Foster, L. J. Proteomics 2009, 72, 12; Munro, S. Cell 2003, 115, 377); however surface labeling of cells requires antibodies, toxins, or nano-scale beads to bind to specific species, which can crosslink them and cause artifactual enrichment (Carvalho, K.; Ramos, L.; Roy, C.; Picart, C. Biophysical Journal 2008, 95, 4348; Dietrich, C.; Volovyk, Z. N.; Levi, M.; Thompson, N. L.; Jacobson, K. Proc. Natl. Acad. Sci. 2001, 98, 10642). In contrast to these methods, some in vitro methods incorporate target species into model membranes such as giant plasma vesicles (Sengupta, P.; Hammond, A.; Holowka, D.; Baird, B. Biochimica et Biophysica Acta (BBA)—Biomembranes 2008, 1778, 20; Levental, I.; Lingwood, D.; Grzybek, M.; Coskun, n.; Simons, K. Proceedings of the National Academy of Sciences 2010), giant unilamellar vesicles (Morales-Penningston, N. F.; Wu, J.; Farkas, E. R.; Goh, S. L.; Konyakhina, T. M.; Zheng, J. Y.; Webb, W. W.; Feigenson, G. W. Biochimica et Biophysica Acta (BBA)—Biomembranes 2010, 1798, 1324; Baumgart, T.; Hunt, G.; Farkas, E. R.; Webb, W. W.; Feigenson, G. W. Biochimica et Biophysica Acta (BBA)—Biomembranes 2007, 1768, 2182), or supported lipid bilayers (Dietrich, C.; Volovyk, Z. N.; Levi, M.; Thompson, N. L.; Jacobson, K. Proceedings of the National Academy of Sciences 2001, 98, 10642; Chiantia, S.; Ries, J.; Kahya, N.; Schwille, P. ChemPhysChem 2006, 7, 2409; Burns, A. R.; Frankel, D. J.; Buranda, T. Biophysical Journal 2005, 89, 1081) to study their partition behavior in carefully controlled conditions. These approaches report the affinity of membrane biomolecules to different lipid domains by their partition coefficients, the concentration distribution of the target molecules among the different phases at the detection time point (Wang, T.-Y.; Leventis, R.; Silvius, J. R. Biophysical Journal 2000, 79, 919). Because the first detection time point occurs long after the molecules have been exposed to the various lipid phases, the partitioning process already begins, or even completes, during the preparation step; therefore, the kinetics of the partitioning process are often missed.

However, information about the kinetics of partitioning process is especially important and relevant to our understanding of membrane organization and function because cells are not in equilibrium (Lingwood, D.; Simons, K. Science 2010, 327, 46; Pike, L. J. Journal of Lipid Research 2006, 47, 1597; Kenworthy, A. K.; Nichols, B. J.; Remmert, C. L.; Hendrix, G. M.; Kumar, M.; Zimmerberg, J.; Lippincott-Schwartz, J. The Journal of Cell Biology 2004, 165, 735) and species are dynamically entering and leaving various regions within the cell membrane.

The current understanding of cell membrane lipid rafts is that they are highly dynamic and transient, and they can sometimes be stabilized to form large microdomains (Pike, L. J. Journal of Lipid Research 2006, 47, 1597; Jacobson, K.; Mouritsen, O. G.; Anderson, R. G. W. Nat Cell Biol 2007, 9, 7; Mishra, S.; Joshi, P. G. Journal of Neurochemistry 2007, 103, 135). How fast a biomolecule can partition into the membrane domain compared to the lifetime of the domain, the kinetic competition of various molecules for a particular microdomain, and the timescale of their association with different phases relative to the timescale of particular biological events are all important to correctly predict cell responses and the regulation of protein activity (Sachs, J. N.; Engelman, D. M. Annu. Rev. Biochem. 2006, 75, 707; Wong, W.; Schlichter, L. J. Biol. Chem. 2003, 279, 444). In the special case of membrane-bound species, recent literature highlights the importance of considering not only structure as a determining factor for membrane protein function, but the coupling of protein dynamics and the interaction with the local lipid environment as well (Sachs, J. N.; Engelman, D. M. Annu. Rev. Biochem. 2006, 75, 707). For a membrane molecule to associate with a patch of lipid domain, it may need to diffuse to the patch, pass across the interface, and diffuse inside the patch. Current methods for studying the dynamic membrane heterogeneity such as single particle techniques and fluorescence correlation spectroscopy (FCS) focus on obtaining the diffusion rate for a molecule in different specified phases or detecting the lifetime of the membrane domains. However, no method has been developed in the art to study the rate of a membrane species passing across a phase interface between the coexistent phases. The challenge comes from the experimental difficulty in defining the phase interface and monitoring the species passing the well-defined interface.

To study the kinetics of a membrane biomolecule associating or dissociating from one membrane phase into another, one needs to critically determine when partitioning starts and monitor the concentration change in different phases in real time. This example describes a microfluidic platform for measuring association/dissociation kinetics that employs patterned lipid membrane phases at pre-defined locations. When membrane biomolecules meet/or leave a particular lipid phase is controlled, and thus partitioning can be monitored from very early times. Using this platform, the early-time association/or dissociation kinetics of membrane-bound species to/from different lipid phases were measured. The kinetic parameters for the partitioning of several membrane-bound species, including an important signaling glycolipid, G_{M1}, were studied.

Domain size and location are difficult to control in conventional model membrane systems, where an unpredictable phase separation process is used to generate membranes with two-phase coexisting regions (Morales-Penningston, N. F.; Wu, J.; Farkas, E. R.; Goh, S. L.; Konyakhina, T. M.; Zheng, J. Y.; Webb, W. W.; Feigenson, G. W. Biochimica et Biophysica Acta (BBA)—Biomembranes 2010, 1798, 1324; Blanchette Craig, D.; Lin, W.-C.; Ratto Timothy, V.; Longo Marjorie, L. Biophysical journal 2006, 90, 4466; Lin, W.-C.; Blanchette, C. D.; Ratto, T. V.; Longo, M. L. In Methods in Membrane Lipids 2007, p 503; Bagatolli, L. A. Biochimica et Biophysica Acta (BBA)—Biomembranes 2006, 1758, 1541; Crane, J. M.; Tamm, L. K. Biophysical Journal 2004, 86, 2965). Because phase separation and biomolecule partitioning are happening concurrently in this conventional approach, it is nearly impossible to track the biomolecule of interest to measure its association kinetics to a particular phase. In contrast to conventional methods, the compositions of stable coexistent phases were first prepared separately as vesicle solutions and then they were patterned together to create a contiguous two-phase supported lipid bilayer within a microfluidic channel network. Supported lipid bilayers and can be formed by lipid vesicle fusion inside microfluidics (Brian, A. A.; McConnell, H. M. Proc. Natl. Acad. Sci. 1984, 81, 6159) and have served as excellent mimics of the cell membrane in numerous applications in these platforms (Groves, J. T.; Dustin, M. L. Journal of Immunological Methods 2003, 278, 19; Richter, R. P.; Him, J. L. K.; Brisson, A. Materials Today (Oxford, United Kingdom) 2003, 6, 32; Sackmann, E. Science (Washington, D. C.) 1996, 271, 43; Chao, L.; Gast, A. P.; Hatton, T. A.; Jensen, K. F. Langmuir 2009, 26, 344; Daniel, S.; Diaz, A. J.; Martinez, K. M.; Bench, B. J.; Albertorio, F.; Cremer, P. S. Journal of the American Chemical Society 2007, 129, 8072). The choices for the two compositions that yield stable phase-separated bilayers are guided by tertiary lipid phase diagrams (Simons, K.; Vaz, W. L. C. Annu. Rev. Biophys. Biomol. Struct. 2004, 33, 269; Veatch, S. L.; Keller, S. L. Phys. Rev. Lett. 2005, 94, 148101), as described below. The two-phase pattern is formed using laminar flow to deliver lipid vesicles of specific compositions to certain regions within the microfluidic channel (Kam, L.; Boxer, S. G. Journal of the American Chemical Society 2000, 122, 12901; Leonenko, Z. V.; Carnini, A.; Cramb, D. T. Biochimica et Biophysica Acta 2000, 1509, 131). Vesicles fuse and the resulting bilayer regions heal together, but do not mix, to form a contiguous two-phase bilayer. Characterization of membrane phase stability is described in the Supplementary Information. The specific membrane-bound biomolecules to assay are fluorescently tagged to visualize and monitor their association with each lipid phase and track changes in their partitioning using a basic inverted fluorescence microscope. Because the locations of the two phases within the membrane are known, no special labeling of the phases themselves is needed and therefore artifacts that could result from labeling are avoided. Next, a hydrodynamic force from the bulk flow in the main microchannel is used to transport the membrane-bound biomolecules along the main channel where they contact the raft phase and can partition into it, as illustrated in FIG. 4. All transport and partitioning takes place within the two-dimensional supported bilayer plane where the two phases meet along a “line” interface. Association kinetics and dissociation kinetics can be decoupled in the measurements and both the kinetic parameters can be obtained.

### 6.1.2. Experimental

Materials

1-Palmitoyl-2-oleoyl-sn-glycero-3-phosphocholine (POPC), cholesterol (Chol) and N-palmitoyl-D-erythro-sphingosylphosphorylcholine (PSM) were purchased from Avanti Polar Lipids (Alabaster, Ala.). N-(4,4-difluoro-5,7-dimethyl-4-bora-3a,4a-diaza-s-indacene-3-propionyl)-1,2-dihexadecanoyl-sn-glycero-3-phosphoethanolamine, triethylammonium salt (head-labeled BODIPY® FL DHPE), BODIPY®FL C5-ganglioside G_{M1} (tail-labeled G_{M1}), and Alexa Fluor® 594 hydrazide was purchased from Invitrogen (Eugene, Oreg.). Bovine brain asialoganglioside-G_{M1} and all other reagents, unless otherwise specified, were purchased from Sigma (St. Louis, Mo.). Glass coverslips (25 mm×25 mm; No. 1.5) from VWR were used as supports for the bilayers. Polydimethylsiloxane (PDMS; Sylgard 184) used to fabricate microfluidic devices was purchased from Robert McKeown Company (Branchburg, N.J.).

Lipids and additives used to make bilayers of various compositions were: 1-Palmitoyl-2- oleoyl-sn-glycero-3-phosphocholine (POPC), cholesterol (Chol) and N-palmitoyl-D-erythro-sphingosylphosphorylcholine (PSM). Three lipids were used in the kinetic studies: N-(4,4-difluoro-5,7-dimethyl-4-bora-3a,4a-diaza-s-indacene-3-propionyl)-1,2-dihexadecanoyl-sn-glycero-3-phosphoethanolamine, triethylammonium salt (BODIPY® FL DHPE), a head group-labeled lipid denoted as BODIPY-DHPE in this work; bovine brain asialoganglioside-G_{M1} labeled with Alexa Fluor® 594 hydrazide, denoted here as head-labeled G_{M1}; and BODIPY®FL-C5 G_{M1}, denoted here as tail-labeled G_{M1}.

Methods

Conjugation of G_{M1} head group with Alexa Fluor®594 label. Alexa Fluor 594 hydrazide-conjugated G_{M1} (head-labeled G_{M1}) was prepared by modifying a method developed by Burns et al. (Burns, A. R.; Frankel, D. J.; Buranda, T. Biophysical Journal 2005, 89, 1081). 1 mg/ml Bovine brain asialoganglioside-G_{M1} was prepared in 100 mM sodium acetate buffer (pH 5.5). 20 mM sodium meta-periodate was added gradually to oxidize the sugar residues in the head group of the G_{M1} to aldehydes. The reaction proceeded for 30 min on ice and protected from light. The suspension was then purified by ultrafiltration in the same buffer using 1 k Microsep™ Centrifugal Devices (VWR) at 14,000 RPM, repeating several times to remove the sodium-meta periodate. 10 mM Alexa Fluor 594 hydrazide was added to the oxidized G_{M1}, spontaneously reacting with aldehydes to form fairly stable hydrazone linkages (Dirksen, A.; Dawson, P. E. Bioconjugate Chemistry 2008, 19, 2543). The reaction was agitated for 3 hr at room temperature. The unreacted dye was removed by using 1 k Microsep™ Centrifugal Devices (VWR), repeating in PBS buffer, until the supernatant was optically clear. The labeled G_{M1} was dried under vacuum and stored as a powder at −80° C.

Preparation of large unilamellar vesicles (LUVs). Large unilamellar vesicles (LUVs) were prepared to form supported lipid bilayers (SLBs) on glass supports within microfluidic devices using the vesicle fusion method. To create the desired composition of SLB, appropriate amounts of lipids and additives were first mixed together in chloroform and then the chloroform was removed by drying under vacuum. The dried lipid material was then reconstituted into multi-lamellar vesicles at a concentration of 2 mg/ml in buffer composed of 5 mM phosphate buffered saline (PBS) with 150 mM NaCl at a pH of 7.4. LUVs were formed by extrusion by passing the reconstituted mixture 19 times through a 50 nm polycarbonate filter in an Avanti Mini-Extruder (Alabaster, Ala.). The vesicle solutions were diluted to 0.5 mg/mL before use.

Vesicle size characterization. A Zetasizer Nano (Malvern Instruments, Worcestershire, UK) was used to determine lipid vesicle size using dynamic light scattering. Plastibrand disposable cuvettes (model 7591 70) were used and samples were equilibrated at 25° C. for two minutes before measurements were taken. All vesicles were on the order of 100 nm in diameter after processing.

Preparation of PDMS microfluidic device. The polydimethylsiloxane (PDMS) microfluidic device was made by soft lithography using methods known in the art. A mold for PDMS casting was fabricated in the following way. First, a silicon wafer was coated with P20 primer and 2 μm SPR220-3 photoresist. The coated wafer was placed under a photomask containing the pattern of the desired microchannel network and exposed to ultraviolet light, according to the standard protocol for this photoresist. After development, the positive photoresist was removed in the areas exposed to the light. The entire substrate was then placed in a silicon wafer plasma etcher (Unaxis SLR 770). Only the region in the silicon wafer where the photoresist had been removed was etched by plasma to the desired height (70 μm for the channel used). The photoresist layer was later washed off in a hot strip bath, leaving the etched silicon wafer as a mold for PDMS channels. PDMS prepolymer, along with a curing agent, was then cast on the mold and cured at 85° C. for 3 hr, producing a soft flexible material with the channels embedded in negative relief once removed from the mold. The channel inlets and outlets connecting to outside tubing are formed by punching the PDMS mold with 20 gauge needles (610 μm).

Glass coverslips, which become the fourth wall of the microfluidic channel, were cleaned in piranha solution (70% H₂SO₄ and 30% H₂O₂) for 10 min, rinsed thoroughly with distilled water for 20 min, and then dried in high purity nitrogen gas. Both the cleaned glass and PDMS mold were treated with oxygen plasma for 30 sec and then gently pressed together to seal the device and form the channels.

Preparation of patterned two-phase coexistent lipid bilayers in a microfluidic device. Supported lipid bilayers (SLBs) are commonly formed by the rupture and self-assembly of large unilamellar lipid vesicles in solution onto glass (Brian, A. A.; McConnell, H. M. Proc. Natl. Acad. Sci. USA 1984, 81, 6159; Johnson, J. M.; Ha, T.; Chu, S.; Boxer, S. G. Biophysical Journal 2002, 83, 3371) or other oxidized supports (Richter, R. P.; Borat, R.; Brisson, A. R. Langmuir 2006, 22, 3497), as illustrated in FIG. 17a. A bilayer is formed during laminar flow conditions instead of under stagnant incubation. Laminar flow is an advantage for patterning heterogeneous bilayers in microfluidic channels because reagents follow streamlines with minimal mixing (Kam, L.; Boxer, S. G. Journal of the American Chemical Society 2000, 122, 12901). Thus, lipid vesicles of different compositions can be sent through the channel on different streamlines and upon rupture will form contiguous, parallel bilayers of different compositions with stable interfaces, if the compositions are chosen so that they are phase stable.

### 6.1.3. Results and Discussion

Formation of contiguous patterned membrane and loading and transport of membrane-bound biomolecules. This example shows that a contiguous patterned membrane can be formed and membrane species transported in it using hydrodynamic flow of the aqueous buffer within in the microfluidic channel. A contiguous lipid membrane is formed with POPC-rich phase (70/20/10 molar ratio of POPC/PSM/Chol), raft phase (60/40 molar ratio of PSM/Chol), and the membrane loaded with 1 mol % of Alexa-594 head-labeled G_{M1} and 1 mol % of head-labeled BODIPY-DHPE doped in the POPC-rich phase. These two molecules are used for this demonstration because G_{M1} is well-known to have high affinity to raft, while BODIPY-DHPE associates with raft to a lesser extent (Burns, A. R.; Frankel, D. J.; Buranda, T. Biophysical Journal 2005, 89, 1081).

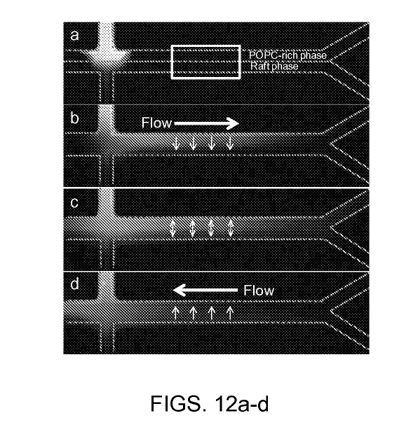

FIG. 12(a) shows the image after patterning the various bilayers in the device before a hydrodynamic force was applied to transport the species in the membrane. The load region contains the mixture of both BODIPY-DHPE, and Alexa-594 head-labeled G_{M1}. Alexa-594 head-labeled G_{M1} is already able to diffuse and partition into the raft phase region in the membrane even before any flow has started.

FIG. 12(b), an applied hydrodynamic flow of the bulk solution in the microchannel, provides a shear stress to drive the lipid membrane on the glass support to move. The use of shear stress from hydrodynamic flow above supported bilayers to move membrane-bound species has been characterized and reported in the literature (Jonsson, P.; Beech, J. P.; Tegenfeldt, J. O.; Hook, F. Journal of the American Chemical Society 2009, 131, 5294). While other methods to transport molecules in supported bilayers are also available (e.g., electrophoresis), hydrodynamic flow was chosen because physiological buffers can be used and are not limited to only assaying charged biomolecules. The fluorescently-labeled species in the POPC-rich phase of this system move approximately ten times faster in the POPC-rich phase than they do in the raft phase under the same hydrodynamic flow conditions. The relative speed difference between the two phases allows the user to view the entire system as a 2-D process in a membrane plane where convective flow of the POPC-rich phase membrane passes the embedded target molecules along the interface of a relatively fixed raft phase.

FIG. 12(c) shows the partitioning after the hydrodynamic flow inside the microchannel was stopped for two hours to allow molecules to penetrate into the raft phase region and reach equilibrium; here it is easy to see the distinct phases divided by a well-defined interface down the middle of the main channel.

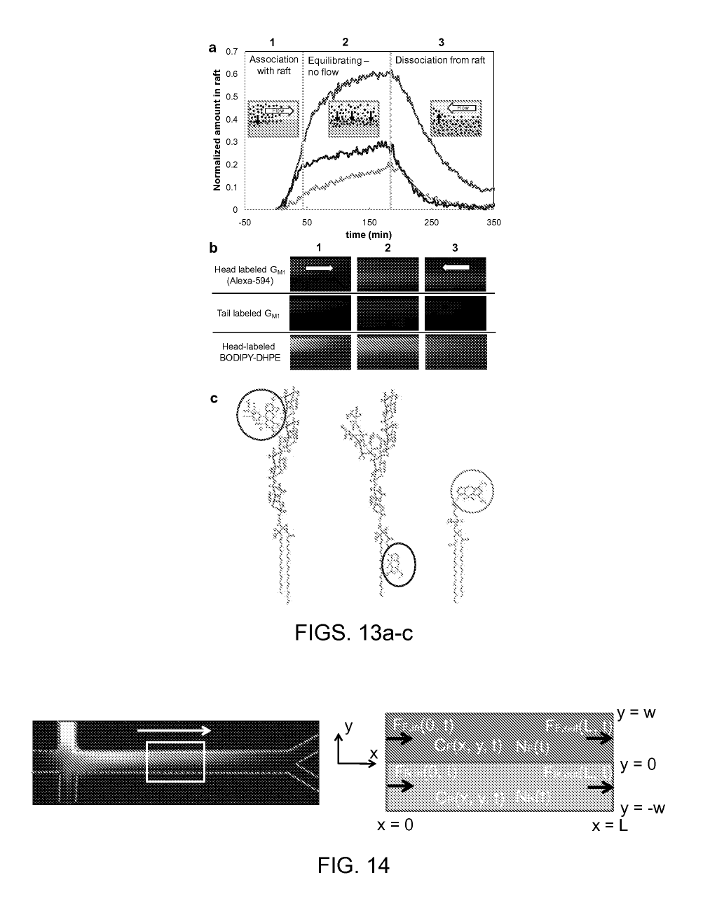

Finally, hydrodynamic flow was induced in the opposite direction, as shown in FIG. 12(d), a convective flow moving the species in the opposite direction was observed. In this image it is also easy to see that the species in the POPC-rich phase bilayer move much faster than those in the raft phase. When the flow is reversed, the POPC-rich phase enriched in BODIPY-DHPE is transported back through the channel much faster than the G_{M1} enriched in the raft phase, even though G_{M1} has a much larger extracellular structure extending into the bulk flow compared to BODIPY-DHPE (see structures in FIG. 13c).

Partitioning kinetic measurements by monitoring species' concentrations in the raft phase with time. It was next demonstrated how to use this platform to measure association/dissociation kinetics by monitoring species' concentrations using fluorescence intensity as a proxy for concentration. The partitioning assay was carried out in three stages: association stage, equilibrium stage, and dissociation stage (FIGS. 13a-c). The experimental procedure and analyses are somewhat analogous to the measurement of adsorption/desorption binding kinetics by surface plasmon resonance (SPR) (Wegner, G. J.; Wark, A. W.; Lee, H. J.; Codner, E.; Saeki, T.; Fang, S.; Corn, R. M. Analytical Chemistry 2004, 76, 5677), except here there is a 2-D planar geometry and observing association/dissociation kinetics of membrane-bound biomolecules to/from a lipid membrane phase along an interface. To obtain association kinetics from POPC-rich phase to raft phase, target species in the POPC-rich phase were brought to a pristine raft phase and the amount entering into the raft phase in the control volume was monitored with time (FIG. 13a, stage 1). After most of the species initially loaded in the channel entered into the main partitioning channel, the next step was to stop the convective flow and wait until the system equilibrated (FIG. 13a, stage 2). Equilibration was confirmed when the concentration profile no longer changed across the channel. Finally, to obtain dissociation kinetics, the net amount of species leaving the raft phase was monitored when the hydrodynamic flow was reversed and fresh POPC-rich phase membrane could be monitored for the re-association of molecules now moving from the raft phase back into the POPC-rich phase (FIG. 13a, stage 3). Three different molecules were tested: Alex594-head-labeled G_{M1}, BODIPY-tail-labeled G_{M1}, and head-labeled BODIPY-DHPE. FIG. 13a shows how their accumulation amount in the raft phase varies with time during the different stages, and FIG. 13b shows a corresponding image observed during each of the association, equilibration, and dissociation stages. FIG. 13c shows the chemical structures of the target biomolecules used in these studies.

Theory of partitioning kinetic analyses. The association/dissociation kinetics of fluorescently-labeled species was analyzed into/from the raft phase using a mass-balance approach. A control volume used for the mass balance is illustrated in FIG. 14. The accumulation of species in the raft phase over time can be represented as:

$$\frac{d {N}_{R}}{d t} = {\int }_{x = 0}^{x = L} {r}_{+} ( {C}_{F} ( x , 0 , t ) , {k}_{+} ) d x - {\int }_{x = 0}^{x = L} {r}_{-} ( {C}_{R} ( x , 0 , t ) , {k}_{-} ) d x + ( {F}_{R , out} ( 0 , t ) - {F}_{R , in} ( L , t ) ) \qquad ( Eq . 1 )$$

where N_{R} is the amount of species in the raft phase region; L is the interface length between the raft phase and the POPC-rich phase; r₊ is the association term, a function of species concentration at the interface in the POPC-rich phase (C_{F}(x,0,t) and association rate constant (k₊); r₋ is the dissociation term depending on the species concentration at the interface in the raft phase (C_{R}(x,0,t)) and dissociation rate constant (k); and F_{R,in} and F_{R,out} are the convective molar flow rates into and out the raft region. The subscripts “R” and “F” refer to the raft and POPC-rich phases, respectively.

Association kinetic analysis. To obtain the association kinetic information from the POPC-rich phase to the raft phase, the experiment was operated in such a way so that only the association term, r₊, is important and the dissociation term and the convective flow rate terms can be neglected in Equation 1. At the beginning of the experiment, a hydrodynamic force was provided to move the species into the control volume (the white box in FIG. 14). The control volume is initially devoid of any fluorophores. The time for species entering into the POPC-rich phase region inside the control volume was set as time zero. When time was close to zero, F_{R,in} and F_{R,out} were still negligible since the species moved much faster (ten times) in the POPC-rich phase than in the raft phase. In addition, at time close to zero, r₋ was also negligible since the concentration in the raft phase was still very low. Next, it was assumed that the association follows first order kinetics in species concentration, so that Equation 1 can be simplified to Equation 2 at beginning of the association stage:

$$\frac{d {N}_{R} ( t )}{d t} = {\int }_{x = 0}^{x = L} {k}_{+} {C}_{F} ( x , 0 , t ) d x \qquad ( Eq . 2 )$$

In the association stage, species concentration at the interface in the POPC-rich phase, C_{F}(x,0,t), varied significantly with both x and t and its integration over x and t is sensitive with the interface boundary chosen. To obtain a more robust way of expressing the integration term, a mass balance for the POPC-rich phase region was applied and the integration term was replaced with a function of the inlet and outlet molar flow rates in the POPC-rich phase. Equation 2 can then be written as Equation 3:

$${N}_{R} ( {t}_{a} ) = \frac{{k}_{+}}{w} {\int }_{t = 0}^{t = {t}_{a}} ( {\int }_{t = 0}^{t = {t}_{a}} ( {F}_{F , in} ( 0 , t ) - {F}_{F , out} ( L , t ) ) d t ) d t \qquad ( Eq . 3 )$$

where F_{F,in} and F_{F,out} are the convective molar flow rates into and out the POPC-rich phase region; w is the width of the POPC-rich phase (as shown in FIG. 14); and tₐ is a certain time after the species started to enter into the control volume. To determine the association rate constant, k₊, from our data, a parameter, a, was defined in Equation 3, and the net amount of species in the raft phase (N_{R}) was plotted against a in FIG. 5. α is defined as:

$$α = 1 / w {\int }_{t = 0}^{t = {t}_{a}} ( {\int }_{t = 0}^{t = {t}_{a}} ( {F}_{F , in} ( 0 , t ) - {F}_{F , out} ( L , t ) ) d t ) d t \qquad ( Eq . 4 )$$

Notice that since Equation 3 is valid only at early times, when the dissociation term and convective flow terms in the raft phase (in Equation 1) are still negligible, only the initial slope of the plot represents k₊.

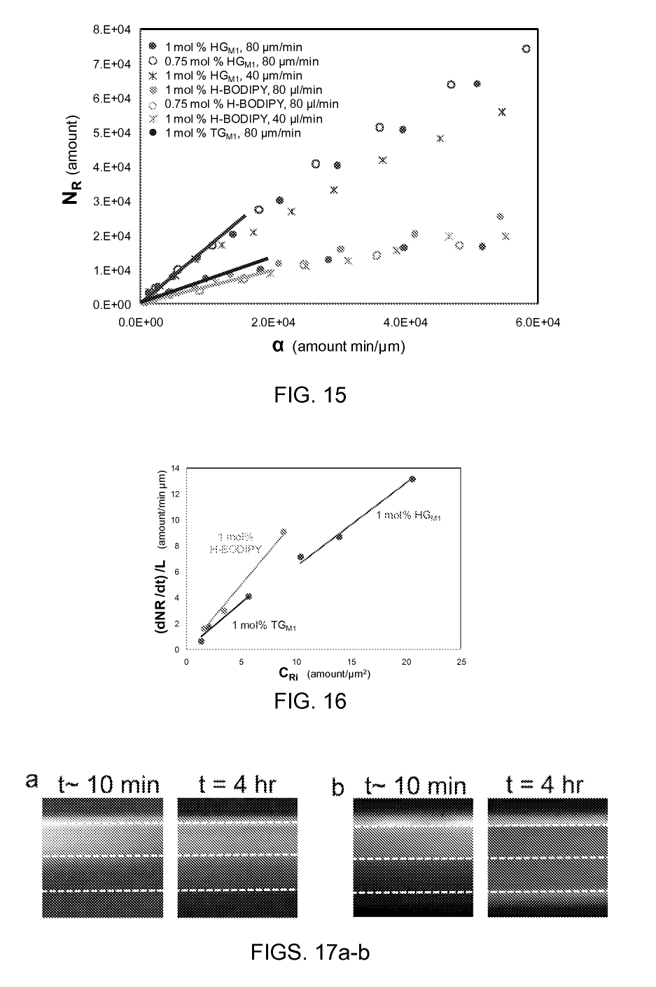

Next, it was checked whether the first order kinetic assumption made in the analysis is valid. When the kinetics are first order in concentration, the amount entering the raft depends on the summation of all of the species concentration approaching the interface. On the other hand, if the kinetics are not first order, the amount entering into the raft phase will depend on the concentration distribution. For example, for a second order association, a sharp concentration distribution causes a higher overall flux of species entering into the raft region than the one a uniform concentration distribution, since the high concentration provides more weight because it is squared. Therefore, to check if the association kinetics are indeed first order with concentration, the initial species concentration and the convection flow rate of the POPC-rich phase was varied to vary the concentration distribution with time in the control volume. In FIG. 15, the curves in the N_{R}-α plot obtained from the systems with different initial species concentrations or convection flow rates have very similar initial slope, indicating that the concentration distribution does not influence the overall amount entering into the raft, and the assumption of first order kinetics is justified.

Dissociation kinetic analyses. To obtain the dissociation kinetic information, the association process was reversed by bringing a pristine POPC-rich phase in contact with the raft phase already loaded with target species (following an equilibration step, described in the next section) and tracked their dissociation. This step was accomplished by reversing the hydrodynamic flow and pushing fresh POPC-rich phase bilayer back through the channel. When the concentration in the POPC-rich phase is low, the association term in Equation 1 may be neglected. In addition, since the system was allowed to equilibrate with no flow for two hours before reversing the flow to conduct the dissociation step, the species concentration in the raft was relatively constant in the x-direction in the control volume. In the middle of the channel, it was assumed that the flow rate of raft phase was relatively constant in the x-direction under the same hydrodynamic force. Therefore, F_{R,in} and F_{R,out} (flow rate times concentration) are similar and the molar flow rate term could be neglected. If it is further assumed that the dissociation also follows first order kinetics in concentration at the raft phase interface, one can simplify Equation 1 to Equation 5 during the late dissociation stage:

$$\frac{d {N}_{R} ( t )}{d t} = - {\int }_{x = 0}^{x = L} {k}_{-} {C}_{R} ( x , 0 , t ) d x \qquad ( Eq . 5 )$$

After the equilibrating stage, the species concentration in the x-direction inside the control volume is relatively uniform. Therefore, it can be assumed that the concentration is independent of x and Equation 5 can be rewritten as:

$$\frac{d {N}_{R} ( {t}_{d} )}{d t} = - {k}_{-} {C}_{Ri} ( {t}_{d} ) L \qquad ( Eq . 6 )$$

where C_{Ri} represents the species concentration at the interface in the raft phase region; L is the length of the control volume; and t_{d} is a certain time during the dissociation measurement.

The left term in Equation 6 can be obtained from the slope of the dissociation part of N_{R}-t plot, such as FIG. 13(a). As for the right term of Equation 6, in contrast to the association case, the species concentration is no longer uniform along the y-direction due to the slow diffusion of species in the raft phase so the bulk concentration cannot be used to represent the interface concentration. Therefore, one needs to obtain C_{Ri} directly from the intensity profile in the y-direction, as described herein. At different time points during the dissociation stage, one can obtain dN_{R}/dt and a corresponding C_{Ri} at that time. The dissociation rate constant, k₋, can be obtained from the slope of the plot of (dN_{R}(t)/dt)/L against C_{Ri}(t), as shown in FIG. 16. The data points used to obtain the dissociation rate constant are chosen at the time when the concentration of species is already low in the POPC-rich phase so that the association term can be neglected and Equation 6 is valid.

Partition coefficients. The partition coefficient is defined as the ratio of the concentration in the raft phase region over the concentration in the POPC-rich phase region after the system has equilibrated for 2 hrs, as shown in the left two terms of Equation 7:

$$K = \frac{{C}_{R}}{{C}_{F}} = \frac{{k}_{+}}{{k}_{-}} \qquad ( Eq . 7 )$$

At equilibrium, the concentrations of species in each phase in the system do not change. At any location at the interface, the rate of species leaving from the raft phase to POPC-rich phase should be equal to the rate of species entering into the raft from the POPC-rich phase at the interface, expressed in Equation 8:

k₊C_{Fi}=k₋C_{Ri} (Eq. 8)

Under equilibrium conditions, one can obtain the right two terms of Equation 7 by reorganizing Equation 8. K, the partition coefficient at equilibrium, was obtained for a target species in two ways.

First, K was obtained by measuring the relative fluorescence intensity in each phase after equilibrating and taking the ratio of raft concentration to POPC-rich concentration. The concentration of each phase at the phase boundaries was used. In separate control experiments, it was verified that the intensity of the fluorescence varied linearly with concentration in the range used and that the fluorescence levels of each biomolecule in the raft and POPC-rich phases were nearly identical.

Second, the K values were obtained from the ratio of the association/dissociation kinetic parameters, which is valid for first order kinetics. This provides a cross-check for the assumption about the order of kinetics. The values obtained using this latter method were consistent with the K's obtained directly from the fluorescence intensity data, as summarized in Table 1. This match further justified the assumption of first order partitioning kinetics that was made to obtain the kinetic parameters initially.

TABLE 1

| | k₊ | k₋ | K | K |
|---|---|---|---|---|
| | (μm/min) | (μm/min) | (=k₊/k₋) | (eq) |
| Head- | 1.53 ± 0.03 | 0.85 ± 0.03 | 1.8 | 1.96 |
| labeled | | | | |
| G_{M1} | | | | |
| Head- | 0.57 ± 0.08 | 0.98 ± 0.04 | 0.58 | 0.6 |
| labeled | | | | |
| BODIPY- | | | | |
| DHPE | | | | |
| Tail- | 0.64 ± 0.07 | 0.72 ± 0.05 | 0.89 | 0.75 |
| labeled | | | | |
| G_{M1} | | | | |

Structure of G_{M1} influences the raft affinity. Tail-labeled G_{M1} shows weaker intrinsic affinity to the raft phase than the head-labeled G_{M1} (Chiantia, S.; Ries, J.; Kahya, N.; Schwille, P. ChemPhysChem 2006, 7, 2409; Burns, A. R.; Frankel, D. J.; Buranda, T. Biophysical Journal 2005, 89, 1081; Wang, T.-Y.; Silvius, J. R. Biophysical Journal 2003, 84, 367). Head-labeled G_{M1} has the same acyl chains as a regular G_{M1}, whose carbonyl and amide functional groups can form hydrogen bonding with other sphingolipids and cholesterol (Barenholz, Y.; Thompson, T. E. Biochimica et Biophysica Acta (BBA)—Reviews on Biomembranes 1980, 604, 129). Hydrogen-bonding enhances its association with the raft phase. When G_{M1} is labeled at its tail, the bulky fluorophore can cause steric hindrance preventing G_{M1} from forming hydrogen bonding with other molecules. These structural distinctions explain differences in overall affinity; these differences also impact the kinetics of association, while dissociation is less affected.

As for head-labeled BODIPY-DHPE, literature has reported that it prefers to partition into disordered phase (Baumgart, T.; Hunt, G.; Farkas, E. R.; Webb, W. W.; Feigenson, G. W. Biochimica et Biophysica Acta (BBA)—Biomembranes 2007, 1768, 2182; Chiantia, S.; Ries, J.; Kahya, N.; Schwille, P. ChemPhysChem 2006, 7, 2409; Burns, A. R.; Frankel, D. J.; Buranda, T. Biophysical Journal 2005, 89, 1081). Although having saturated acyl chains, which might lead to predictions that it would associate with the raft phase over the more disordered POPC-rich phase, it lacks the functional groups to form hydrogen bonding network with the molecules in the raft phase (Ramstedt, B.; Slotte, J. P. Biochimica et Biophysica Acta (BBA)—Biomembranes 2006, 1758, 1945). Apparently these differences can impact BODIPY-DHPE's association with the raft phase.

Time scale for membrane molecules to associate/dissociate with “lipid rafts”. In this example, the obtained association/dissociation rate constants represent how fast a membrane species passes across a designated interface to/from the raft phase to/from the POPC-rich phase. The current understanding of cell membrane lipid rafts are that they are small (10-200 nm), heterogeneous, highly dynamic, sterol- and sphingolipid-enriched domains, and they can sometimes be stabilized to form large microdomains (Pike, L. J. Journal of Lipid Research 2006, 47, 1597; Jacobson, K.; Mouritsen, O. G.; Anderson, R. G. W. Nat Cell Biol 2007, 9, 7; Kenworthy, A. K.; Nichols, B. J.; Remmert, C. L.; Hendrix, G. M.; Kumar, M.; Zimmerberg, J.; Lippincott-Schwartz, J. The Journal of Cell Biology 2004, 165, 735). For a membrane molecule to associate with a patch of lipid rafts, it needs to diffuse to the patch, jump across the interface, and diffuse inside the patch. The rate limiting step(s) determines how fast a molecule can associate and interact with a new phase.

The interfacial mass transfer kinetic process across the interface could be the rate limiting process of the overall association process of a biomolecule with a particular lipid phase under certain conditions. Fluorescence recovery after photobleaching (FRAP) was used to measure the diffusivity of Alexa594-head-labeled G_{M1} in both lipid phases separately and found the diffusion coefficients to be 0.77 μm²/sec and 0.06 μm²/sec in the POPC-rich and raft phases respectively. To make a rough estimate of when this interfacial rate might be limiting, the following scenario was considered. G_{M1} molecule must diffuse to a domain with 200 nm size from a location at 1 μm ( 1/20 of a 20 gm cell size) in the POPC-rich phase away from the domain. The rate for a molecule to diffuse to the interface can be estimated to be 0.77 μm/sec (diffusion coefficient/distance). The rate of the molecule to pass across the interface is ˜0.025 gm/sec (from Table 1) and the diffusion rate from the interface into the middle of the raft patch (˜100 nm) is estimated as 0.6 μm/sec. These approximate calculations show that the kinetics of molecule passing the interface could play an important role or dominate in the overall association rate of G_{M1} to the raft region. This method could also be applied to describe the association of a bundle of crosslinked molecules, or membrane molecules with lipid shells after measuring their partitioning kinetics and diffusivity in each phase.

As a final note, assays were performed within a supported lipid bilayer. It is known that the solid support interaction can influence the lipid mobility in a supported lipid bilayer. In general, the diffusivity of species in supported lipid bilayers is slower than in giant unilamellar vesicles which have free standing bilayers (Przybylo, M.; Sykora, J.; Humpolikova, J.; Benda, A.; Zan, A.; Hof, M. Langmuir 2006, 22, 9096). It is possible that the rate of species passing the interface is also influenced to the same degree by the support and the rate-limiting step analyses is still valid. In addition, although it is not known how the solid support interaction might influence the absolute number of the association/dissociation rate constants, the relative affinity between different membrane species should still be valid as long as the species does not have specific or direct interaction with the solid support. In the case of glycolipids or lipid-linked proteins, e.g., GPI-linked, this assumption should be valid; however, for transmembrane proteins, to minimize these effects, a cushion or spacing layer (e.g., dextran or polyethylene glycol) could be used to minimize protein-support interactions.

This example demonstrates the possibility to construct a predetermined patterned two-phase coexistent bilayer. The two-phase coexistent compositions chosen were guided by previously published phase diagrams for giant unilamellar vesicles and a hypothetical tie line. The phase diagram in a supported bilayer is likely to be shifted somewhat from the giant unilamellar case (Tokumasu, F.; Jin, A. J.; Feigenson, G. W.; Dvorak, J. A. Biophysical Journal 2003, 84, 1); however the compositions that were used were found empirically to be two-phase stable for at least four hours. This method can be extended to test the relative association affinity of biomolecules to different types of physiologically-relevant phases with more complicated membrane compositions, as long as the phases are sufficiently stable during the time needed for the assay.

### 6.1.4. Conclusions

This example demonstrates a method for assaying the partition kinetics of head-labeled G_{M1}, tail-labeled G_{M1}, and head-labeled BODIPY-DHPE in patterned, heterogeneous supported lipid bilayers. These different molecules exhibit significantly different partitioning kinetics to the raft phase tested here. Structural features of these biomolecules, such as label location and the ability to form hydrogen bonds, may influence the partitioning kinetics, especially the association of these molecules to the raft phase, based on these results. Dissociation kinetics from the raft phase are much less variable between these different biomolecules and may indicate that these features have less influence on the dissociation behavior.

The general features of the BDP described in this example that enable the user to make these kinetic measurements are (1) control of when a target biomolecule first encounters predetermined and (2) fixed phase locations, which do not require extra labels to distinguish different phases from each other. In this case, fluorescent labels were used only to track target species and to quantify the amount of species entering/leaving the pre-defined raft region. In other embodiments, other surface characterization tools that can measure spatial mass change, such as surface plasma resonance imaging (Nelson, B. P.; Grimsrud, T. E.; Liles, M. R.; Goodman, R. M.; Corn, R. M. Analytical Chemistry 2000, 73, 1), and quartz crystal microbalance (Ebara, Y.; Okahata, Y. Journal of the American Chemical Society 1994, 116, 11209; Keller, C. A.; Kasemo, B. Biophysical Journal 1998, 75, 1397) can be coupled to the BDP for label free measurement. In addition, the approach described herein could be extended to assay the partition behavior of other lipids and proteins with posttranslational modifications, such as the addition of GPI anchors, sterols, and single saturated or unsaturated fatty acids, since creating supported bilayers from sections of cell membrane is already possible (Dodd, C. E.; Johnson, B. R. G.; Jeuken, L. J. C.; Bugg, T. D. H.; Bushby, R. J.; Evans, S. D. Biointerphases 2008, 3, FA59; Frankel, D. J.; Pfeiffer, J. R.; Surviladze, Z.; Johnson, A. E.; Oliver, J. M.; Wilson, B. S.; Burns, A. R. Biophysical Journal 2006, 90, 2404).

Many factors have been suggested to regulate the raft phase association of membrane biomolecules, such as the different type of lipid modifications (Lopez-Montero, I.; Monroy, F.; Velez, M.; Devaux, P. F. Biochimica et Biophysica Acta 2010, 1798, 1348), changes in raft composition (Wong, W.; Schlichter, L. J. Biol. Chem. 2003, 279, 444; Tikku, S.; Epshtein, Y.; Collins, H.; Travis, A. J.; Rothblat, G. H.; Levitan, I. Am. J. Physiol. Cell Physiol. 2007, 293, 440), chemical exposure (Dolganiuc, A.; Bakis, G.; Kodys, K.; Mandrekar, P.; Szabo, G. Alcoholism: Clinical and Experimental Research 2006, 30, 76), changes in pH/ionic strength (Hartmann, W.; Galla, H. J.; Sackmann, E. FEBS Letters 1977, 78, 169), small molecule binding and crosslinkers (Kahya, N.; Brown, D. A.; Schwille, P. Biochemistry 2005, 44, 7479). The BPD and the analysis method disclosed herein can be used to test and understand the influence of how structural and environment factors influence molecules' partition kinetics to the raft phase, which may provide insight into how the partition dynamics of cell membrane species can be altered.

### 6.1.5. Supplementary Materials and Methods

The compositions of the raft phase and POPC-rich phase used in this example were chosen based on a published tertiary phase diagram of POPC/PSM/Chol (Edidin M. Lipids on the Frontier: A Century of Cell-Membrane Bilayers. Nature. 2005; 4:414-8; Simons K, Ikonen E. Functional Rafts in Cell Membranes. Nature. 1997; 387:569-72). The phase diagram, illustrated in FIG. 2b, highlights the two-phase coexistent region in gray. A hypothetical tie line in this phase diagram is plotted, guided by previous literature (Simons K, Ikonen E. Functional Rafts in Cell Membranes. Nature. 1997; 387:569-72; Sprenger R R, Horrevoets J G. The Ins and Outs of Lipid Domain Proteomics. Proteomics. 2007; 7:2895-903). Compositions were picked close to the ends of a hypothetical tie line. These compositions are 70/20/10 molar ratio of POPC/PSM/Chol and 60/40 molar ratio of PSM/Chol for POPC-rich and raft phases respectively. A composite membrane could be patterned based on these compositions inside a microfluidic channel using laminar flow (as will be described in detail next) to define regions of specific lipid phases within the channel. Membrane-bound biomolecules were able to move between the phases after patterning and that different biomolecules had various levels of affinity for the various phases. These patterned phases were stable for at least four hours, while a membrane patterned in the same way, but using the same composition for both regions, did not have the same effect (FIGS. 17a-b). This latter result shows that the enriching effect observed was not an artifact of the manner in which the bilayers were patterned in the channel.

To form a composite lipid bilayer with raft phase, POPC-rich phase, and load membrane at specified locations, as illustrated in FIGS. 5a-f, the following procedure was used. First, raft phase vesicles were sent from port 5 to 1 and concurrently sent a buffer stream from port 4 to 1 to keep raft phase vesicles located in half of the channel (FIG. 5b). During this step, the system was heated to 65° C. (both the device and the raft mixture), so that the raft phase lipid mixture was above its phase transition temperature and readily fused to glass surface to form a bilayer. Afterwards, a 65° C. buffer was used to rinse out the excess vesicles and the system was equilibrated to room temperature for 1 hr to allow the raft membrane gradually cool down. Second, the vesicles with load mixture (mixture denoted as light and dark dots in FIG. 5c) were sent from port 2 to 3. The load mixture was composed of the same composition as the POPC-rich phase, but also included small amounts of the glycolipid, G_{M1}, and BODIPY DHPE lipid (approx. 1 mol % of each). At the same time, buffer streams from 1, 4, and 5 to 3 were maintained to prevent the load vesicles from entering into the main channel. The membrane with the load mixture formed only on the glass surface where there was no bilayer under the stream of the load vesicles. Third, the POPC-rich phase vesicles were sent through the main channel and filled the exposed regions of the glass surface which had not been covered by lipid membranes (FIG. 5d). The formed composite membrane is shown in FIG. 5e.

In all of these steps, the vesicles were exposed to the glass surface for 5 min under flow and then rinsed with buffer for 20 min. When the raft phase and POPC-rich phase formed, the flow rates of vesicle solutions and rinsing buffers were kept at 20 μL/min in the main channel (100 μm wide and 70 μm high). When the load formed, the flow rate of the load vesicle solution in the upstream side channel (50 μm wide and 70 μm high) was 10 μL/min and the overall flow rate of the load vesicle solution and buffers in the downstream side channel (50 μm wide and 70 μm high) was 30 μL/min.

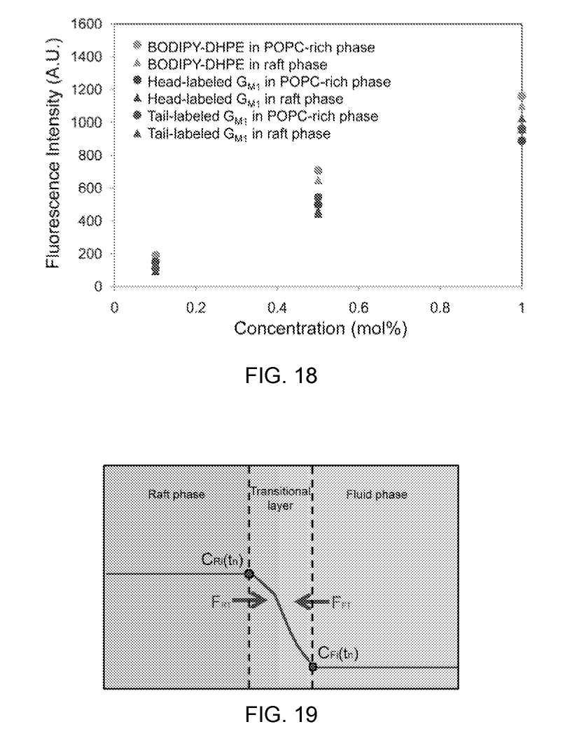

Fluorescence intensity versus concentration of fluorescent molecules. The data analyses in the next section are based on the fluorescence intensity. In general, the fluorescence intensity is proportional to the labeled molecule's concentration for low concentrations (Shaw A S. Lipid Rafts: Now You See Them, Now You Don't. Nature Immunology. 2006; 7:1139-42). The nonlinearity that occurs at high concentration is usually due to fluorescence quenching. This range is avoided. The highest concentration use in this example was 1 mol % head-labeled G_{M1}, 1 mol % of BODIPY-DHPE and 1 mol % of G_{M1} tail-labeled with BODIPY. FIG. 18 shows that the method was operating at the concentration range where the fluorescence intensity is proportional to the concentration. In addition, the fluorescence intensity of the fluorescent species is the same in the POPC-rich phase or in raft phase when the samples have the same species concentration so that one can use intensity as a reporter for concentration.

Equipment Setup

Microscope Configuration. An inverted Zeiss Axiovert Observer. Z1 fluorescence microscope equipped with a Plan-Apochromat 10× or 5× objectives, a Hamamatsu EM-CCD camera (ImageEM, model C9100-13, Bridgewater, N.J.), and X-Cite® 120 microscope light source (Lumen Dynamics Group Inc., Canada) were used to carry out imaging. ET GFP filter cube (49002, c106273, Chromatech Inc.) was used to collect the fluorescence emitted from BODIPY-DHPE or BODIPY labeled G_{M1}, ET MCH/TR filter cube (49008, c106274, Chromatech Inc.) was used to collect the fluorescence emitted from Alexa®594-GM1. For fluorescence recovery after photobleaching experiments (FRAP), a CVI Melles Griot argon-krypton tunable laser (Model 643-AP-A01) was coupled into the microscope.

Image Processing. Zeiss axiovision software was used to obtain images and the fluorescence intensity data for lipid diffusion and kinetic data analyses. The contrast of an entire image was enhanced in Image) (NIH, Bethesda, Md.) when necessary.

Lipid Diffusion Measurements. Diffusion coefficients of all biomolecule targets in each phase were determined by fluorescence recovery after photobleaching (FRAP). To measure the diffusion coefficient and mobile fraction of the biomolecule in a supported bilayer, liposomes of the same composition used in experiments were prepared with 0.1% of either BODIPY DHPE or G_{M1}. 0.5 mg/ml liposomes were incubated in a PDMS well attached to a piranha cleaned slide. Lipid vesicles were allowed to incubate for 5 minutes, before being rinsed with buffer for one minute. In the case of the POPC-rich phase bilayer, the entire process is at room temperature. In the case of the raft phase bilayer, the prepared vesicle solutions were heated to 65° C. (a temperature above the miscibility transition temperature of the lipid mixture). The warm vesicle solutions were added to wells heated to 65° C. and were later rinsed with the buffer at 65° C. Following the formation of the raft phase bilayer, the heated sample was gently cooled down to room temperature prior measuring the diffusion coefficient.

To aid in focusing on the plane of the bilayer on the microscope, the bilayer was scratched with a dissection tool to remove a thin line of bilayer. Following the scratching step, the bilayer was rinsed again for one minute with buffer to remove any lipids in the bulk that were removed by scratching. A 20 μm diameter spot in the supported lipid bilayer was bleached with a laser for 200 ms. The recovery of the photobleached spot was recorded for 10 minutes for the POPC-rich phase sample and one hour for the raft phase sample. The fluorescence intensity of the bleached spot was determined after background subtraction and normalization for each image. The recovery data was fit using the method of Soumpasis (Zheng Y Z, Foster L J. Biochemical and Proteomic Approaches for the Study of Membrane Microdomains. Proteomics. 2009; 72:12-22). The diffusion coefficient, D, was then calculated using the following equation: D=w²/4t_{1/2}, where w is the full width at half-maximum of the Gaussian profile of the focused beam. The average diffusion coefficient and mobile fractions were determined from three separate samples of each sample and are listed in Table 2. These values are in line with numerous reports of lipid diffusion in supported bilayers.

TABLE 2
Diffusion coefficients of head-labeled BODIPY-DHPE, head-labeled
G_{M1} and tail-labeled G_{M1} in different phases.

| | BODIPY- | Head-labeled | Tail-labeled |
|---|---|---|---|
| Lipid Phase | DHPE | G_{M1} | G_{M1} |
| POPC-rich phase | 0.63 μm²/s | 0.77 μm²/s | 0.82 μm²/s |
| (70/20/10 POPC/PSM/Chol) | | | |
| Raft phase | 0.05 μm²/s | 0.06 μm²/s | 0.08 μm²/s |
| (60/40 PSM/Chol) | | | |

Data Analyses

Interfacial region between the two phases from the experimental data. Ideally, there should be a sharp physical interface between the two phases. However, in reality, the measurement of fluorescence intensity did not change abruptly but continually between two phases, causing difficulty in defining the single physical interface of the two phases. One of the traditional interfacial models, the transitional layer model of Guggenheim (Simons K, Vaz WLC. Model Systems, Lipid Rafts, and Cell Membranes. Annu Rev Biophys Biomol Struct. 2004; 33:269-95; Pike L J. Rafts defined. J Lipid Research. 2006; 47:1597-8), can be used to explain what was observed, as illustrated in FIG. 19. At the region where the two phases contact, there is a gradual mixing or organization change of phase materials, causing an interfacial layer instead of a single interface plane. A continual property change occurs in this transitional layer.

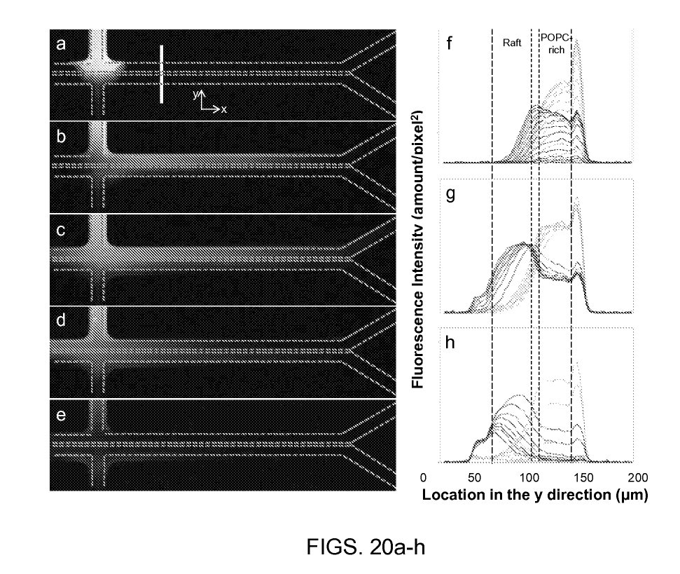

FIGS. 20a-h show a bird's eye view fluorescent images and fluorescence intensity profile across the channel during the association stage, equilibrating stage and dissociation stage. (a) The image before any hydrodynamic force was applied to move head-labeled G_{M1} and head-labeled BODIPY-DHPE. The POPC-rich phase was loaded in the top part of the main channel while the raft phase was in the bottom half. Both of these phases were initially devoid of fluorophore; the interface boundaries between the phases and the boundary of the microchannel are marked by dashed lines that have been superimposed on the image. (b) Association stage: a hydrodynamic force was applied to the right to bring the target species into the main chamber. (c) Equilibrating stage: the hydrodynamic force was stopped and the system was allowed to sit for 2 hrs to equilibrate. (d) Dissociation stage: a reversed hydrodynamic flow was applied to move the target species away from the main chamber. (e) The final state after most of the species were removed from the sight of view. (f) Fluorescence intensity profile change with time along the line denoted in (a) during the association stage. The time interval between each profile was 2 min. (g) Intensity profile change during the equilibrating stage. The time interval was 20 min. (h) Intensity profile change during the dissociation stage. The time interval was 10 min.

The interface region was defined based on the fluorescence intensity profile at the equilibrating stage, as shown in FIG. 20g. The right boundary line between the POPC-rich phase and the transitional layer was defined at the location where BODIPY-DHPE fluorescence intensity drops and head-labeled G_{M1}'s intensity rises significantly. The profile on the right in the POPC-rich phase is relatively flat, indicating where the bulk POPC-rich phase is. The left interface boundary between the raft phase and transitional layer was defined primarily based on the intensity profile of head-labeled G_{M1}. Next, it was assumed that the location of these interface boundaries defined in equilibrating stage did not change significantly in our association and dissociation stages. In fact, there were consistent kinks in the intensity profiles when these boundaries were superimposed to the intensity profiles in the association and dissociation stages (FIGS. 20f and h). The kinks in the intensity profile, sudden changes of intensity gradient, may imply the target species' mobility or diffusion change and these property changes support the location of these interface boundaries, where the content or organization of phase materials start to vary from those in the bulk phase (O'Malley M A, Lazarova T, Britton Z T, Robinson A S. High-Level Expression in Saccharomyces Cerevisiae Enables Isolation and Spectroscopic Characterization of Functional Human Adenosine A2A Receptor. J Struct Biol. 2007; 159:166-78; Simons K, Vaz W L C. Model Systems, Lipid Rafts, and Cell Membranes. Annu Rev Biophys Biomol Struct. 2004; 33:269-95; Pike L J. Rafts defined. J Lipid Research. 2006; 47:1597-8).

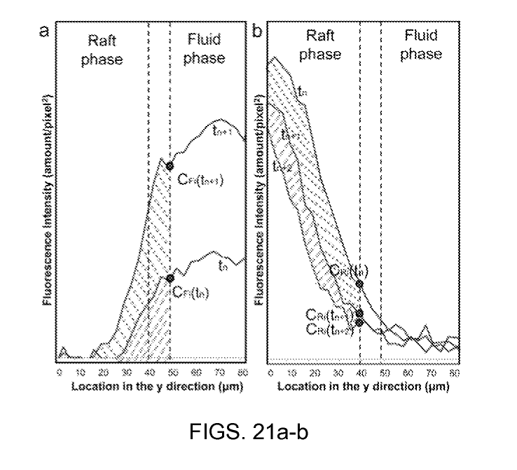

According to theory, the association or dissociation kinetic parameters should be based on the species concentration at the interface. In these experiments and in reality, a transitional interface layer is obtained instead of an interface plane, and the parameters obtained are based on the concentrations measured at the boundaries of the interface layer. FIGS. 21a-b illustrate how the concentration changes in the y-direction during the association and dissociation stages. As discussed in the next section, the obtained association kinetic parameter was based on the species concentration at the interface boundary at the POPC-rich phase side, defined as C_{Fi}, and the flux entering into the transitional layer and the bulk raft phase from the bulk POPC-rich phase. On the other hand, the dissociation kinetic parameter was based on the species concentration at the interface boundary at the raft phase side, defined as C_{Ri}, and the flux leaving from the raft phase to the transitional layer and the bulk POPC-rich phase.

As shown in FIG. 19, the net species passing rate from the bulk raft phase region to the transitional layer is defined as F_{RT} and the net species passing rate from the bulk POPC-rich phase to the transitional layer is defined as F_{FT}. At equilibrium, the species concentration inside the transitional layer should be at steady state and the two rates should be the same:

F_{RT}=F_{FT} (Eq. S1)

First order association and dissociation is assumed, and the net passing rate through the interface boundary is proportional to the concentration at the boundary. Therefore, Equation S1can be rewritten as Equation S2:

k₊C_{Fi}=k₋C_{Ri} (Eq. S2)

where k₊ and k₋ are the association rate constant and dissociation rate constant for the first order association and dissociation.

The equilibrium constant was defined as the ratio of the bulk phase concentrations, as shown in the left part of Equation S3:

$$K = \frac{{C}_{R}}{{C}_{F}} = \frac{{k}_{+}}{{k}_{-}} \qquad ( Eq . S 3 )$$

At equilibrium, it can be assumed that the concentrations at the interface boundaries are similar to the concentrations in the bulk phases. Therefore, the right term in Equation S3 can be obtained from Equation S2. In this example, K (the equilibrium partition coefficient) of a target species was obtained by using the measured fluorescence intensity in interface region of each phase. The K numbers obtained from the ratio of kinetic parameters (described next) were found to be consistent with the K directly from the fluorescence intensity, as reported in Table 1 above, supporting the data analyses to obtain the kinetic parameters.

Derivation of Association Kinetic Equation. To obtain Equation 3 from Equation 2 in the main text, a mass balance for the POPC-rich phase region in the control volume was applied:

∫_{y=0}^{y=w} ∫ₓ₌₀^{x=L} C_{F}(x, y, t=tₐ)dxdy=∫₌₀^{t=t}^{ₐ}(F_{F,in}(0, t)<F_{F,out}(L, t))dt+∫ₜ₌₀^{t=t}^{ₐ} ∫ₓ₌₀^{x=L} (r₋−r₊)dxdt (Eq. S4)

where w is the width of the POPC-rich phase region, L is the length of the POPC-rich phase region, tₐ represents a certain time after the species entering into the control volume, F_{F,in} and F_{F,out} are the convective molar flow rates into and out the POPC-rich region, r₊ is the association term representing the amount leaving the POPC-rich phase to the raft phase, and r₋ is the dissociation term representing the amount entering into the POPC-rich phase from the raft phase.

At the beginning of partitioning, the amount entering the raft phase from the POPC-rich phase, the third term in Equation S4 is negligible compared to the amount in the POPC-rich phase. In addition, FIG. 21 a shows that the fluorescence intensity profile in the POPC-rich phase across the channel (in the y-direction) is relatively flat in the POPC-rich phase during the association step, indicating that C_{F} is independent of y-direction, so the species concentration at the interface, C_{Fi}, can be represented as C_{F}. Therefore, Equation S4 can be written as Equation S5, correlating overall interface concentration in the POPC-rich phase to the inlet and outlet molar flow rates in the POPC-rich phase:

$${\int }_{x = 0}^{x = L} {C}_{Fi} ( x , t = {t}_{a} ) d x ∼ \frac{1}{w} {\int }_{t = 0}^{t = {t}_{a}} ( {F}_{F , in} ( 0 , t ) - {F}_{F . out} ( L , t ) ) d t \qquad ( Eq . S 5 )$$

Substituting Equation S5 into Equation 2 and integrating over time, the accumulated amount in the raft phase was obtained as a function of the inlet and outlet molar flow rates in the POPC-rich phase:

$${N}_{R} ( {t}_{a} ) = \frac{{k}_{+}}{w} {\int }_{t = 0}^{t = {t}_{a}} ( {\int }_{t = 0}^{t = {t}_{a}} {F}_{F , in} ( 0 , t ) - {F}_{F . out} ( L , t ) d t ) d t \qquad ( Eq . S 6 )$$

The initial slope represents k₊, since the equation is valid only at beginning when the dissociation term and convective flow term are still negligible. To check if the association kinetics are first order with concentration, the initial species concentration and the convection flow rate of the POPC-rich phase was varied to vary the concentration distribution with time in the control volume. As discussed above, initial slopes of the N_{R}-α plot obtained from the systems with different initial species concentrations or convection flow rates were very similar, indicating that the concentration distribution does not influence the overall amount entering into the raft, and our assumption of first order kinetics is justified. The k₊, or the initial slopes, of these different systems are listed in Table 3.

TABLE 3
The association rate constant (k₊) from different experiments.

| | Concentration: | Concentration: | Concentration: |
|---|---|---|---|
| | 1 mol % | 1 mol % | 0.75 mol % |
| | Flow rate: | Flow rate: | Flow rate: |
| | 80 μl/min | 40 μl/min | 80 μl/min |
| Head-labeled | 1.53 ± 0.03 | 1.50 | 1.55 |
| G_{M1} | | | |
| Tail-labeled G_{M1} | 0.72 ± 0.07 | N/A | N/A |
| BODIPY-DHPE | 0.57 ± 0.08 | 0.58 | 0.49 |

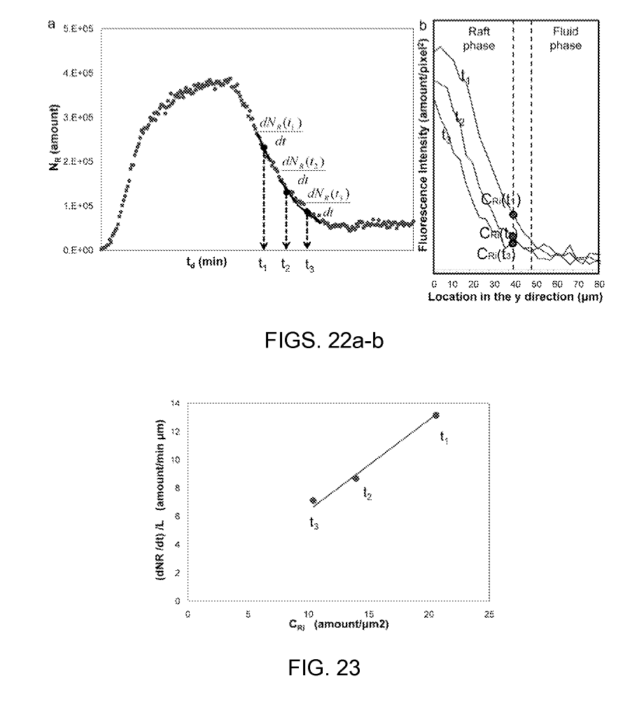

Dissociation Kinetics. As disclosed above, the mass balance equation of the raft phase region during the dissociation stage can be written as Equation S7:

$$\frac{d {N}_{R} ( {t}_{d} )}{d t} = - {k}_{-} {C}_{Ri} ( {t}_{d} ) L \qquad ( Eq . S 7 )$$

where C_{Ri} represents the species concentration at the interface boundary of the raft phase; L is the length of the interface; and t_{d} is a certain time during the dissociation measurement. The left term of Equation S7 was obtained from the slope of the dissociation part of the N_{R}-t curve, as illustrated in FIG. 22(a). As for the right term, different from the association case, the species concentration is no longer uniform along the y-direction and the bulk concentration cannot be used to represent the interface concentration, due to the slow diffusion of species in the raft phase. Therefore, C_{Ri} was obtained from the intensity profile in the y-direction as a function of time, as shown in FIG. 22b. At a certain time point during the dissociation stage, dN_{R}/dt and the corresponding C_{Ri} was obtained. The dissociation rate constant, k₋, can be obtained from the slope of the plot of (dN_{R}(t)/dt)/L against C_{Ri}(t), as shown in FIG. 23. The data points used to obtain the dissociation rate constant were chosen at the time when the concentration of species is already low in the POPC-rich phase so that the association term can be neglected and Equation S7 is valid.

### 6.2 Example 2

### Microfluidic-Based Approach for Studying Lipid-Protein Interactions

Introduction

This example discloses an approach for using an embodiment of the BPD to study the protein-lipid interface, define key interactions for protein activity, and study dynamic shifts in partitioning behavior when subjected to stimuli, all of which can lead to better understanding of specific interactions important for normal health and in disease.

Increasing evidence suggests that specific lipid-protein interactions are required for membrane protein activation. Unfortunately, current technology and biochemical methods are limited in their ability to interrogate the lipid-protein biointerface and its impact on the activity of the protein, but this information is vital to understanding healthy cellular processes and diagnosing disease.

The BPD exploits the natural partitioning behavior of lipids and proteins into phase-separated regions of a heterogeneous biomimetic membrane, and then using a chemical or environmental trigger, elicits a change in partitioning to initiate mixing and interaction of the previously segregated species. Species are selected to be included in the biomimetic platform to aid in reducing the complexity of real cell membranes, while still preserving important features of cell membranes like two-dimensional fluidity and phase separation. Within this platform it is easy to control chemical composition and spatial organization of heterogeneous patterns, and detect, sort, and quantify interactions between species. This example describes the impact of specific lipids and microenvironments on a model protein, Human Placental Alkaline Phosphatase (PLAP). Developing a convenient interaction assay is not only important for understanding basic cellular functions and disease processes, but also crucial for the design of reliable screening tools for accurate drug interactions and diagnostic tools that utilize membrane proteins. This technology can impact a number of fields in which membrane lipid alterations affect protein function and disease processes, such as diabetes and atherosclerosis.

Effect of external stimuli on protein partitioning into lipid microdomains. The user can control when proteins and lipids interact, which is important for later decoupling how changes in these interactions impact protein activity. Proteins derived from biological membranes are incorporated into the platform and partitioned into specific bilayer regions. Stimuli that alter partitioning of membrane lipids and proteins are characterized. A model protein is used: a commercially available protein, human placental alkaline phosphatase (PLAP). Stimuli known to impact partitioning can include, but are not limited to: β-cyclodextrin (βCD) exposure, salt and pH gradients, and ligand binding. Changes are tracked using fluorescence and ability to initiate when proteins and lipids interact using external stimuli is characterized.

Detection and quantification of protein activities in different lipid microenvironments. PLAP shows increased activity when residing in specific lipid microenvironments. Biophysical studies show that enzymatic activity of PLAP increases in the absence of cholesterol. Cholesterol efflux is initiated using βCD. To detect and quantify PLAP activity, fluorometric assay is used. The use of the BPD for studying protein-lipid interactions is assessed and the impact of the interaction on protein activity. Stable membrane microenvironments are created in a controlled in vitro system. This approach provides a tractable experimental system in which to study the interactions of myriad membrane species. The BPD circumvents many artifacts and limitations of other methods, has the capability to track transient changes in partitioning, and offers the possibility to assay protein functionality in one convenient platform.

By using the BPD, the user can: 1) directly visualize the partitioning of protein species into micro-domains; 2) control this partitioning by dynamically modulating bilayer micro-domain lipid content; and 3) thereby determine the direct impact of chemicals, compounds and ligands on protein activity.

The BPD can be used to provide a controlled, experimentally tractable native-like membrane environment to which specific stimuli can be introduced under physiological conditions. Fluorescence activity reporters can be used to quantify the dependence of protein activity on membrane environment. Supported lipid bilayers (SLBs) are well-established cell membrane mimics used in many in vitro assays and biosensing applications.

The formation of adjacent bilayers of stable compositions is straightforward. At microfluidic dimensions, laminar flow conditions prevail, i.e. streamlines do not mix turbulently.

Another advantage of the device is its planar geometry, which makes it easy to couple to an inverted fluorescence microscope for monitoring the transient changes in partitioning behavior of the constituents,

Effect of external stimuli on protein partitioning into lipid microdomains. Proteins are incorporated into the BPD, partitioned into specific membrane compositions within the device, and external stimuli are used to induce changes in partitioning behavior to control when certain species can interact. In this example, glycolipid G_{M1}, and the enzyme PLAP were chosen. βCD exposure, salt or pH gradients, and ligand or antibody binding were chosen as stimuli.

PLAP is used as an exemplary protein target in this example because 1) it is commercially available; 2) it can be fluorescently-labeled (Kahya N, Brown D A, Schwille P. Raft Partitioning and Dynamic Behavior of Human Placental Alkaline Phosphatase in Giant Unilamellar Vesicles. Biochemistry. 2005; 44:7479-89); 3) it has established differences in enzymatic activity based on its local lipid environment, which are readily detectable with commercially available assay kits (Sesana S, Re F, Bulbarelli A, Salerno D, Cazzaniga E, Masserini M. Membrane Features and Activity of GPI-Anchored Enzymes: Alkaline Phosphatase Reconstituted in Model Membranes. Biochemistry. 2008; 47:5433-40); and 4) it is GPI lipid-linked (Lehto M T, Sharom F J. Proximity of the protein moiety of a GPI-anchored protein to the membrane surface: a FRET study. Biochemistry. 2002; 41:8368-76), which ensures it will remain mobile in the SLB and should have a preferred partitioning in raft phase regions (Paulick M G, Bertozzi C R. The Gylcosylphosphatidylinositol Anchor: A Complex Membrane-Anchoring Strucutre for Proteins. Biochemistry. 2008; 47:6991-7000). G_{M1} is used in this example because 1) G_{M1} fluorescently labeled on the external sugars can be synthesized; 2) this variant of G_{M1} can be partitioned into raft micro-domains on the BPD.

βCD-mediated efflux is used to disrupt raft microdomains and stimulate a change in G_{M1} partitioning. Changes in partitioning of both G_{M1} and PLAP can also be induced by binding to CTB and/or antibodies (Kahya N, Brown D A, Schwille P. Raft Partitioning and Dynamic Behavior of Human Placental Alkaline Phosphatase in Giant Unilamellar Vesicles. Biochemistry. 2005; 44:7479-89). Antibody binding is known to cause several-fold enrichment in the raft phase of both targets and detection will be straightforward using fluorescence labels or conjugates. Such enrichment was observed in the BPD when CTB binds to G_{M1}.

Incorporating proteins derived from biological membranes into specific membrane regions of the platform. Liposomes are created that contain purified proteins (proteoliposomes) as delivery vehicles to incorporate protein into SLBs following routine vesicle fusion procedures (Brian A A, McConnell H M. Allogenic Stimulation of Cytotoxic T Cells by Supported Planar Membranes. Proc Natl Acad Sci. 1984; 81:6159-63; Keller C A, Kasemo B. Surface specific kineitcs of lipid vesicle adsoprtion measured with a quartz crystal micorbalance. Biophys J. 1998; 75:1397-402; Johnson J M, Ha T, Chu S, Boxer S G. Early steps of supported bilayer fomration probed by single vesicel fluorescence assays. Biophys J. 2002; 83:3371-9; Bakash M M, Dean C, Pautot S, DeMaria S, Isacoff E, Grove J T. Neuronal Activation of GPI-linked Neuroligin-1 Displayed in Synthetic Lipid Bilayer Membranes. Langmuir.21:10693-8). Commercially-available PLAP is incorporated into proteoliposomes using standard procedures (Kahya N, Brown D A, Schwille P. Raft Partitioning and Dynamic Behavior of Human Placental Alkaline Phosphatase in Giant Unilamellar Vesicles. Biochemistry. 2005; 44:7479-89; Sesana S, Re F, Bulbarelli A, Salerno D, Cazzaniga E, Masserini M. Membrane Features and Activity of GPI-Anchored Enzymes: Alkaline Phosphatase Reconstituted in Model Membranes. Biochemistry. 2008; 47:5433-40; 68; Ronzon F, Morandat S, Roux B, Bortolato M. Insertion of a glycophosphatidylinositol-anchored enzyme into liposome. J Membr Biol. 2004; 197:169-77; Angrand M, Briolay A, Ronzon F, Roux B. Detergent-mediated reconsititution of a glycosyl-phosphatidylinositol-protein into liposomes. Eur J Biochem. 1997; 250:168-76; Schroeder R J, London E, Brown D A. Interactions between saturated acyl chains confer detergent resistance on lipids and glycosylphosphatidylinositol (GPI)-anchored proteins: GPI-anchored proeins in liposomes and cells show similar behavior. Proc Natl Acad Sci. 1994; 91(12130-12134)).

Enrichment of the protein is achieved using immuno-purification and relative purity will be quantified using standard SDS-PAGE and densitometric measurements. Fluorescent tags are incorporated on the protein to detect their locations in the device and monitor partitioning behavior into specific membrane regions. A screen is performed for partitioning into liquid ordered regions composed of mixtures of cholesterol and sphingomyelin relative to fluid phase bilayers composed of mainly phospholipids. The presence of the protein in the bilayers is verified using standard fluorescence techniques and their mobility verified using fluorescence recovery after photobleaching (Axelrod D, Koppel D E, Schlessinger J, Elson E, Webb W W. Mobility measurement by analysis of fluorescence photobleaching recovery kinetics. Biophys J. 1976; 19:1055-69) and observing their redistribution into heterogeneous bilayers.

While the process of making proteoliposomes is well-established for GPI-linked proteins (Kahya N, Brown D A, Schwille P. Raft Partitioning and Dynamic Behavior of Human Placental Alkaline Phosphatase in Giant Unilamellar Vesicles. Biochemistry. 2005; 44:7479-89; Sesana S, Re F, Bulbarelli A, Salerno D, Cazzaniga E, Masserini M. Membrane Features and Activity of GPI-Anchored Enzymes: Alkaline Phosphatase Reconstituted in Model Membranes. Biochemistry. 2008; 47:5433-40; Ronzon F, Morandat S, Roux B, Bortolato M. Insertion of a glycophosphatidylinositol-anchored enzyme into liposome. J Membr Biol. 2004; 197:169-77; Angrand M, Briolay A, Ronzon F, Roux B. Detergent-mediated reconsititution of a glycosyl-phosphatidylinositol-protein into liposomes. Eur J Biochem. 1997; 250:168-76; Schroeder R J, London E, Brown D A. Interactions between saturated acyl chains confer detergent resistance on lipids and glycosylphosphatidylinositol (GPI)-anchored proteins: GPI-anchored proeins in liposomes and cells show similar behavior. Proc Natl Acad Sci. 1994; 91(12130-12134)) (of which PLAP is one) mobility of the other membrane spanning proteins may be reduced. A cushion can be used to reduce strong protein interaction with the support (Floyd D L, Ragains J R, Skehel J J, Harrison S C, van Oijen A M. Single-particle kinetics of influenza virus membrane fusion. Proc Natl Acad Sci. 2008; 105:15382-7; Albertorio F, Daniel S, Cremer P S. Supported Lipopolymer Membranes as Nanoscale Filters: Simultaneous Protein Recognition and Size-Selection Assays. J Am Chem Soc. 2006; 128:7168-9; Elender G, Kuhner M, Sackmann E. Functionalisation of Si/SiO2 and glass surfaces with ultrathin dextran films and deposition of lipid bilayers. Biosens & Bioelect. 1996; 11:565-77).

Characterizing Stimuli that Induce Dynamic Changes in Partitioning Behavior.

This experiment demonstrates control over when sequestered lipids interact with mobile proteins, or vice versa, by changing the partitioning behavior using external stimuli. G_{M1} and PLAP both have known stimuli that change their partitioning behavior. Targets are labeled with fluorophores and their partitioning into different bilayer regions is tracked and quantified using fluorescence intensity, as described herein.

Changing the partitioning of G_{M1} from raft phase to a less-ordered fluid phase is accomplished by flowing βCD through the device. βCD removes cholesterol from bilayers and induces a change in the equilibrium of the phases allowing G_{M1} to migrate into the non-raft region (Dietrich C, Volovyk Z N, Levi M, Thompson N L, Jacobson K. Partitioning of Thy-1, GM1, and Cross-linked Phospholipid Analogs into Lipid Rafts Reconstituted in Supported Model Membrane Monolayers. Proc Natl Acad Sci. 2001; 98:10642-7; Ilangumaran S, Hoessli D C. Effects of cholesterol depletion by cyclodextrin on the sphingolipid microdomains of the plasma membrane. Biochem J. 1998; 335:433-40). Partitioning of fluorescently-labeled G_{M1} (or other species) into each bilayer composition is monitored by following the intensity of the fluorescence in each membrane region.

A change in the enrichment of PLAP in the raft phase is accomplished using antibody binding (Kahya N, Brown D A, Schwille P. Raft Partitioning and Dynamic Behavior of Human Placental Alkaline Phosphatase in Giant Unilamellar Vesicles. Biochemistry. 2005; 44:7479-89). Fluorescent labeling of PLAP allows one to monitor its partitioning behavior before and after antibody binding occurs, so its enrichment in the raft phase following binding can be assessed. Fluorescent labeling of the antibody with a different fluorophore allows one to distinguish unbound from bound species and their preferred locations in the device. The impact of antibody binding on enzyme activity has been quantified previously (Brennan C, Christianson K, Surowy T, Mandecki W. Modulation of enzyme activity by antibudy binding to an alkaline phosphatase-epitope hybrid protein. Protein Engineering. 1994; 7:509-14).

Detection and quantification of protein activities in different lipid microenvironments. The BPD can be used to investigate how interactions with local lipids impact protein activity.

Measuring the activity of PLAP. Biophysical studies suggest that enzymatic activity of PLAP increases in the absence of cholesterol (Sesana S, Re F, Bulbarelli A, Salerno D, Cazzaniga E, Masserini M. Membrane Features and Activity of GPI-Anchored Enzymes: Alkaline Phosphatase Reconstituted in Model Membranes. Biochemistry. 2008; 47:5433-40). The impact of different lipid microenvironments on PLAP activity can be assessed; activity is predicted to be lower in the raft phase.

PLAP activity is tested in both kinds of bilayers separately as controls. PLAP is incorporated into the BPD and changes in partitioning are induced using two strategies: 1) βCD exposure, which will induce a change in partitioning of the protein from raft to fluid phase, and 2) antibody binding, which will conversely increase PLAP's enrichment in the raft phase. To detect and quantify changes in PLAP enzymatic activity after either of these stimuli, a commercially available fluorometric assay (e.g., Starbright Alkaline Phosphatase Detection Kit #MT-1000, Sigma-Aldrich, St. Louis, Mo.) can be used.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A biomolecule partitioning device (BPD) for partitioning biomolecules comprising:
a substrate;
a microfluidic channel on the substrate;
a plurality of stable coexistent lipid phases, wherein the surface of the microfluidic channel is coated with a plurality of stable, coexistent lipid phases, thereby forming a supported lipid bilayer.

2. The BPD of claim 1 wherein the biomolecules are cell membrane species.

3. The BPD of claim 1 wherein the plurality of stable coexistent lipid phases comprises a raft-like or a fluid-like lipid composition.

4. The BPD of claim 1 wherein the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

5. The BPD of claim 1 wherein the plurality of stable coexistent lipid phases are patterned within the microfluidic channel.

6. The BPD of claim 1 comprising a cushion for reducing strong protein interaction with the surface of the microfluidic channel.

7. The BPD of claim 1 wherein the supported lipid bilayer comprises at least two different stable, coexistent lipid phases in controllable or preselected spatial or temporal geometries.

8. The BPD of claim 1 wherein an electric field or fluidic flow is applied to the biomolecule.

9. A method for separating biomolecules comprising the steps of:
providing a microfluidic channel;
providing a supported lipid bilayer comprising a plurality of stable coexistent lipid phases, wherein the supported lipid bilayer is patterned within the microfluidic channel;
introducing the biomolecules into the supported lipid bilayer;
applying an electric field or hydrodynamic flow to move the biomolecules through the supported lipid bilayer;
separating migrating biomolecules based on their preference for heterogeneous regions in the supported lipid bilayer; and
collecting the separated biomolecules.

10. The method of claim 9 wherein the biomolecules are separated into a phospholipid-containing region.

11. The method of claim 10 wherein the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

12. A method for sorting biomolecules comprising the steps of:
introducing the biomolecules into a supported lipid bilayer;
patterning a supported lipid bilayer comprising a plurality of stable coexistent lipid phases;
applying an electric field or hydrodynamic flow to move the biomolecules through the supported lipid bilayer;
sorting migrating biomolecules based on their preference for heterogeneous regions in the supported lipid bilayer;
collecting sorted biomolecules in a quantification area; and
classifying the sorted biomolecules based on their affinity for a particular lipid phase.

13. The method of claim 12 wherein the biomolecules are sorted into a phospholipid-containing region.

14. The method of claim 13 wherein the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

15. The method of claim 12 comprising the step of determining the ratio of biomolecules collected in raft versus fluid phases.

16. A method for assaying biomolecule partitioning preference comprising the steps of:
introducing biomolecules into a supported lipid bilayer;
patterning a supported lipid bilayer comprising a plurality of stable coexistent lipid phases;
applying an electric field or hydrodynamic flow to move the biomolecules through the supported lipid bilayer; and
determining the partitioning of the migrating biomolecules based on their preference for heterogeneous regions in the supported lipid bilayer over time.

17. The method of claim 16 wherein the biomolecules are sorted into a phospholipid-containing region.

18. The method of claim 17 wherein the phospholipid-containing region is a raft phase or raft-like region, a 1-Palmitoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

19. A method for assaying interaction of a first biomolecule with a second biomolecule comprising the steps of:
providing a microfluidic channel;
providing a supported lipid bilayer comprising a plurality of stable coexistent lipid phases, wherein the supported lipid bilayer is patterned within the microfluidic channel;
introducing the first cell membrane species into the first phase of two phases of the supported lipid bilayer; and
applying a stimulus to induce mixing and/or biomolecule partitioning preference in the first membrane species positioned in the first phase such that interactions take place with the second cell membrane species positioned in the second phase.

20. The method of claim 19 wherein the first or the second biomolecule is sorted into a phospholipid-containing region.

21. The method of claim 20 wherein the phospholipid-containing region is a raft phase or raft-like region, a 1-Palm itoyl-2-oleoyl-sn-glycero-3-phosphocholine (POPC)-rich region, a cholesterol (Chol) -rich region or a N-palmitoyl-D-erythro-sphingosylphosphorylcholine (PSM)-rich region.

22. The method of claim 19 wherein the stimulus is (or results from) binding of a small molecule, a pH change, a temperature change, an ionic strength change, a chemical stimulus or an electrical stimulus.

23. The method of claim 19 comprising the step of monitoring or determining a change in location of the first or second biomolecule.

24. The method of claim 19 comprising the step of monitoring or determining a change in activity of the first or second biomolecule.

25. The method of claim 19 comprising the step of monitoring or determining a change in function of the first or second biomolecule.

26. The method of claim 9, 12 or 16 wherein the biomolecules are cell membrane species.

27. The method of claim 19 wherein the first or second biomolecule is a cell membrane species.

28. The method of claim 9, 12, 16 or 19 wherein the plurality of stable coexistent lipid phases are patterned within the microfluidic channel.

29. The method of claim 9, 12, 16 or 19 wherein the supported lipid bilayer comprises at least two different stable, coexistent lipid phases in controllable or preselected spatial or temporal geometries.

30. The method of claim 9, 12, 16 or 19 comprising the step of applying an electric field or fluidic flow to the biomolecules.
